(19) United States
(12) Patent Application Publication
(10) Pub. No.: US 2022/0153834 A1
(43) Pub. Date: May 19, 2022

(54) TSG-6 ANTIBODIES AND USES THEREFOR

(71) Applicant: UNIVERSITY HEALTH NETWORK, Toronto (CA)

(72) Inventors: Richard Brokx, Toronto (CA); Jacqueline M. Mason, Toronto (CA); Mark R. Bray, Toronto (CA)

(21) Appl. No.: 17/434,368

(22) PCT Filed: Mar. 11, 2020
(86) PCT No.: PCT/CA20/50321

§ 371 Date: Aug. 26, 2021

Related U.S. Application Data
(60) Provisional application No. 62/817,152, filed on Mar. 12, 2019.

(51) Int. Cl. C07K 16/28; A61P 35/00

(52) U.S. Cl. CPC C07K 16/2803; A61K 2039/505; A61P 35/00

(57) ABSTRACT

The invention provides novel anti-TSG-6 antibodies, pharmaceutical compositions comprising such antibodies, and therapeutic methods of using such antibodies and pharmaceutical compositions for the treatment of diseases such as cancer or autoimmune disease.

### CROSS REFERENCE TO RELATED APPLICATION

The present application claims the benefit of priority to U.S. Provisional Application No. 62/817,152, filed Mar. 12, 2019, the contents of which is expressly incorporated herein in its entirety for all purposes.

### SEQUENCE LISTING

The instant application contains a Sequence Listing that has been submitted electronically and in ASCII format and is hereby incorporated by reference in its entirety. Said ASCII copy, created Mar. 6, 2020, is named 011506-5023_ST25.txt and is 71 kilobytes in size.

### TECHNICAL FIELD

The present disclosure relates to molecules that specifically bind to TSG-6, e.g., human TSG-6 (hTSG-6), and pharmaceutical compositions comprising such TSG-6-binding antibodies thereof. Methods of using the antibodies of the invention to detect human TSG-6 or to modulate human TSG-6 activity in the treatment of various diseases, including autoimmune diseases, inflammatory diseases, fibrotic diseases and cancer, are also encompassed by the invention.

### BACKGROUND OF THE INVENTION

Tumor necrosis factor-inducible gene 6 protein (TSG-6), encoded in humans by the tumor necrosis factor, alpha-induced protein 6 (TNFAIP6) gene, is an extracellular (secreted) protein that belongs to a class of hyaluronan-binding proteins called hyaladherins. TSG-6 contains an N-terminal Link domain, which binds hyaluronan, and a C-terminal CUB domain, which is a multifunctional domain found in many proteins involved in protein-protein interactions. TSG-6 plays a role in modulating immune responses. In general, the protein is considered to be an anti-inflammatory mediator although effects may vary by context; mice in which the gene encoding TSG-6 was disrupted displayed faster progression and worse severity in an arthritis model, but displayed attenuated inflammation and decreased airway eosinophilia in a model of allergic asthma. The apparent main function of TSG-6 is to bind and covalently modify hyaluronan, thus affecting extracellular matrix structure and function; TSG-6 can also bind and modulate the activity of various chemokines, and other extracellular matrix-associated molecules such as chondroidin sulfate, aggrecan, versican, fibronectin and pentraxin-3.

Hyaluronan (hyaluronic acid, or HA), is an extracellular matrix glycosaminoglycan molecule comprised of repeating glucuronic acid and N-acetyl glucosamine subunits. It is thought to provide lubrication and assist motion between adjacent tissue layers, and it is also highly hydrated, thus serving to increase hydrostatic pressure in the extracellular matrix and providing resistance to compression. HA is also a signaling molecule, influencing cellular processes by binding its cell-surface receptors, including CD44, receptor for hyaluronan-associated motility (RHAMM, also known as HMMR or CD168), and lymphatic vessel endothelial hyaluronan receptor 1 (LYVE1). HA is present at high abundance in the stroma of many tumors, including pancreatic cancer, some breast cancers, and other cancers, where it provides a microenvironment favorable for tumor growth, angiogenesis, and metastasis, and is involved in increasing matrix stiffness and hydrostatic pressure, forming physical barrier to entry by immune cells and therapeutic drugs. HA is also present at high amounts in many fibrotic diseases, where it plays similar roles.

TSG-6 is primarily secreted by fibroblasts, smooth muscle cells, and mesenchymal stem cells (MSCs), bone marrow-derived fibroblast-like cells with potent immunosuppressive properties that can extravasate at sites of inflammation. TSG-6 catalyzes the transfer of HC proteins (also known as serum hyaluronan-associated protein or SHAP) from IαI to HA in a transesterification reaction. The resulting HC-HA complex forms cable-like structures rather than more diffuse networks. These cables have altered properties including adhesion of leukocytes via the HA receptor CD44 and altering the polarization of macrophages to the alternatively activated ‘M2’ phenotype. HC-HA cables are thought to play a pathogenic role in cancer; HC-HA has also been detected in excised lung tissue from patients with idiopathic pulmonary artery hypertension, a condition frequently arising from underlying lung fibrosis. TSG-6 also binds and non-covalently crosslinks HA in the absence of HC modification; this HA crosslinking binds leukocytes and maintains them in an unactivated state.

Collectively, these findings suggest that the development of agents useful in inhibiting the HC-HA transfer activity of TSG-6 would be of great benefit in diseases involving high fibroblast activity and extracellular matrix content, including chronic inflammatory diseases, fibrotic diseases, and cancer.

### SUMMARY OF THE INVENTION

In one aspect, the present invention relates to novel anti-TSG-6 antibodies. In some embodiments, the anti-TSG-6 antibodies include a heavy chain variable region comprising an amino acid sequence of SEQ ID NO:1 and a light chain variable region comprising an amino acid sequence of SEQ ID NO:2. In some embodiments, the anti-TSG-6 antibodies include a heavy chain variable region comprising an amino acid sequence of SEQ ID NO:3 and a light chain variable region comprising an amino acid sequence of SEQ ID NO:4. In some embodiments, the anti-TSG-6 antibodies include a heavy chain variable region comprising an amino acid sequence of SEQ ID NO:5 and a light chain variable region comprising an amino acid sequence of SEQ ID NO:6. In some embodiments, the anti-TSG-6 antibodies include a heavy chain variable region comprising an amino acid sequence of SEQ ID NO:7 and a light chain variable region comprising an amino acid sequence of SEQ ID NO:8. In some embodiments, the anti-TSG-6 antibodies include a heavy chain variable region comprising an amino acid sequence of SEQ ID NO:9 and a light chain variable region comprising an amino acid sequence of SEQ ID NO:10. In some embodiments, the anti-TSG-6 antibodies include a heavy chain variable region comprising an amino acid sequence of SEQ ID NO:11 and a light chain variable region comprising an amino acid sequence of SEQ ID NO:12. In some embodiments, the anti-TSG-6 antibodies include a heavy chain variable region comprising an amino acid sequence of SEQ ID NO:13 and a light chain variable region comprising an amino acid sequence of SEQ ID NO:14. In some embodiments, the anti-TSG-6 antibodies include a heavy chain variable region comprising an amino acid sequence of SEQ ID NO:15 and a light chain variable region comprising an amino acid sequence of SEQ ID NO:16. In some embodiments, the anti-TSG-6 antibodies include a heavy chain variable region comprising an amino acid sequence of SEQ ID NO:17 and a light chain variable region comprising an amino acid sequence of SEQ ID NO:18. In some embodiments, the anti-TSG-6 antibodies include a heavy chain variable region comprising an amino acid sequence of SEQ ID NO:19 and a light chain variable region comprising an amino acid sequence of SEQ ID NO:20. In some embodiments, the anti-TSG-6 antibodies include a heavy chain variable region comprising an amino acid sequence of SEQ ID NO:21 and a light chain variable region comprising an amino acid sequence of SEQ ID NO:22. In some embodiments, the anti-TSG-6 antibodies include a heavy chain variable region comprising an amino acid sequence of SEQ ID NO:23 and a light chain variable region comprising an amino acid sequence of SEQ ID NO:24. In some embodiments, the anti-TSG-6 antibodies include a heavy chain variable region comprising an amino acid sequence of SEQ ID NO:25 and a light chain variable region comprising an amino acid sequence of SEQ ID NO:26.

In some embodiments, the anti-TSG-6 antibodies include a vhCDR1 comprising SEQ ID NO:27, a vhCDR2 comprising SEQ ID NO:28, a vhCDR3 comprising SEQ ID NO:29, a vlCDR1 comprising SEQ ID NO:30, a vlCDR2 comprising SEQ ID NO:31, and a vlCDR3 comprising SEQ ID NO:32. In some embodiments, the anti-TSG-6 antibodies include a vhCDR1 comprising SEQ ID NO:33, a vhCDR2 comprising SEQ ID NO:34, a vhCDR3 comprising SEQ ID NO:35, a vlCDR1 comprising SEQ ID NO:36, a vlCDR2 comprising SEQ ID NO:37, and a vlCDR3 comprising SEQ ID NO:38. In some embodiments, the anti-TSG-6 antibodies include a vhCDR1 comprising SEQ ID NO:39, a vhCDR2 comprising SEQ ID NO:40, a vhCDR3 comprising SEQ ID NO:41, a vlCDR1 comprising SEQ ID NO:42, a vlCDR2 comprising SEQ ID NO:43, and a vlCDR3 comprising SEQ ID NO:44. In some embodiments, the anti-TSG-6 antibodies include a vhCDR1 comprising SEQ ID NO:45, a vhCDR2 comprising SEQ ID NO:46, a vhCDR3 comprising SEQ ID NO:47, a vlCDR1 comprising SEQ ID NO:48, a vlCDR2 comprising SEQ ID NO:49, and a vlCDR3 comprising SEQ ID NO:50. In some embodiments, the anti-TSG-6 antibodies include a vhCDR1 comprising SEQ ID NO:51, a vhCDR2 comprising SEQ ID NO:52, a vhCDR3 comprising SEQ ID NO:53, a vlCDR1 comprising SEQ ID NO:54, a vlCDR2 comprising SEQ ID NO:55, and a vlCDR3 comprising SEQ ID NO:56. In some embodiments, the anti-TSG-6 antibodies include a vhCDR1 comprising SEQ ID NO:57, a vhCDR2 comprising SEQ ID NO:58, a vhCDR3 comprising SEQ ID NO:59, a vlCDR1 comprising SEQ ID NO:60, a vlCDR2 comprising SEQ ID NO:61, and a vlCDR3 comprising SEQ ID NO:62. In some embodiments, the anti-TSG-6 antibodies include a vhCDR1 comprising SEQ ID NO:63, a vhCDR2 comprising SEQ ID NO:64, a vhCDR3 comprising SEQ ID NO:65, a vlCDR1 comprising SEQ ID NO:66, a vlCDR2 comprising SEQ ID NO:67, and a vlCDR3 comprising SEQ ID NO:68. In some embodiments, the anti-TSG-6 antibodies include a vhCDR1 comprising SEQ ID NO:69, a vhCDR2 comprising SEQ ID NO:70, a vhCDR3 comprising SEQ ID NO:71, a vlCDR1 comprising SEQ ID NO:72, a vlCDR2 comprising SEQ ID NO:73, and a vlCDR3 comprising SEQ ID NO:74. In some embodiments, the anti-TSG-6 antibodies include a vhCDR1 comprising SEQ ID NO:75, a vhCDR2 comprising SEQ ID NO:76, a vhCDR3 comprising SEQ ID NO:77, a vlCDR1 comprising SEQ ID NO:78, a vlCDR2 comprising SEQ ID NO:79, and a vlCDR3 comprising SEQ ID NO:80. In some embodiments, the anti-TSG-6 antibodies include a vhCDR1 comprising SEQ ID NO:81, a vhCDR2 comprising SEQ ID NO:82, a vhCDR3 comprising SEQ ID NO:83, a vlCDR1 comprising SEQ ID NO:84, a vlCDR2 comprising SEQ ID NO:85, and a vlCDR3 comprising SEQ ID NO:86. In some embodiments, the anti-TSG-6 antibodies include a vhCDR1 comprising SEQ ID NO:87, a vhCDR2 comprising SEQ ID NO:88, a vhCDR3 comprising SEQ ID NO:89, a vlCDR1 comprising SEQ ID NO:90, a vlCDR2 comprising SEQ ID NO:91, and a vlCDR3 comprising SEQ ID NO:92. In some embodiments, the anti-TSG-6 antibodies include a vhCDR1 comprising SEQ ID NO:93, a vhCDR2 comprising SEQ ID NO:94, a vhCDR3 comprising SEQ ID NO:95, a vlCDR1 comprising SEQ ID NO:96, a vlCDR2 comprising SEQ ID NO:97, and a vlCDR3 comprising SEQ ID NO:98. In some embodiments, the anti-TSG-6 antibodies include a vhCDR1 comprising SEQ ID NO:99, a vhCDR2 comprising SEQ ID NO:100, a vhCDR3 comprising SEQ ID NO:101, a vlCDR1 comprising SEQ ID NO:102, a vlCDR2 comprising SEQ ID NO:103, and a vlCDR3 comprising SEQ ID NO:104.

In some embodiments, the anti-TSG-6 antibodies described herein bind human and/or mouse TSG-6.

In some embodiments, the anti-TSG-6 antibodies described herein include a constant region with an amino acid sequence at least 90% identical to a human IgG. In some embodiments, the IgG is selected from an IgG1, IgG2, IgG3 or IgG4. In some embodiments, the IgG is an IgG1. In some embodiments, the IgG is an IgG2b.

In another aspect, the present invention relates to a nucleic acid composition encoding any one of the anti-TSG-6 antibodies described herein. In some embodiments, the nucleic acid composition includes a first nucleic acid comprising the heavy chain, and a second nucleic acid comprising the light chain.

Another aspect of the present invention relates to an expression vector composition that includes any one of the nucleic acid compositions encoding any one of the anti-TSG-6 antibodies described herein. In some embodiments, the first nucleic acid is contained in a first expression vector and the second nucleic acid is contained in a second expression vector. In some other embodiments, the first nucleic acid and the second nucleic acid are contained in a single expression vector.

Another aspect of the present invention relates to a host cell that includes any one of the expression vectors described herein. Also presented is a method of making anti-TSG-6 antibodies, and the method includes culturing the host cell under conditions wherein the antibodies expressed, and recovering the antibodies.

In another aspect, the present invention relates to a composition that includes any one of the anti-TSG-6 antibodies described herein, and a pharmaceutical acceptable carrier or diluent.

Also described is a method of modulating an immune response in a subject, and the method includes administering to the subject an effective amount of any one of the anti-TSG-6 antibodies described herein, or any one of the compositions described herein. In some embodiments, the method stimulates an immune response in the subject and the method includes administering to the subject an effective amount of an anti-TSG-6 antibody, or a composition thereof, that acts as a TSG-6 antagonist. In some embodiments, the method suppresses an immune response in the subject and the method includes administering to the subject an effective amount of an anti-TSG-6 antibody, or a composition thereof, that acts as a TSG-6 agonist.

In further embodiments and in accordance with any of the above, the present disclosure describes a method of treating cancer in a subject comprising administering to the subject an effective amount of an antibody where the antibody serves as a TSG-6 antagonist. In some embodiments, the cancer being treated has high expression of TSG-6 and/or HC-HA. In further embodiments, the subject to be treated has high expression of TSG-6 and/or HC-HA. In some embodiments, the cancer is a solid tumor. In further embodiments, the cancer is melanoma. In some embodiments, the antibody is combined with one or more additional therapeutic agents to treat cancer. In further embodiments, the additional therapeutic agents are other immune checkpoint inhibitors. In some embodiments, the immune checkpoint inhibitors are selected from the group consisting of a PD-1 inhibitor, a PD-L1 inhibitor, a CTLA-4 inhibitor, a TIM-3 inhibitor, and a LAG-3 inhibitor.

In another aspect, the present invention relates to a method of treating fibrosis in a subject comprising administering to the subject an effective amount of an antibody, wherein the antibody serves as a TSG-6 antagonist. In some embodiments, the fibrotic tissue has high expression of TSG-6 and/or HC-HA. In some embodiments the subject to be treated has high expression of TSG-6 and/or HC-HA. In some embodiments, the antibody is combined with one or more additional therapeutic agents to treat fibrosis.

In another aspect, the present invention relates to a method of treating an autoimmune or inflammatory disorder in a subject comprising administering to the subject an effective amount of an antibody, wherein the antibody serves as a TSG-6 antagonist. In some embodiments, the autoimmune or inflammatory disorder is idiopathic pulmonary artery hypertension. In some embodiments, the subject has high expression of TSG-6 and/or HC-HA. In some embodiments, the subject also has lung fibrosis. In some embodiments, the antibody is combined with one or more additional therapeutic agents to treat pulmonary artery hypertension and/or lung fibrosis. In some embodiments, the autoimmune or inflammatory disorder is asthma. In some embodiments, the subject to be treated has high expression of TSG-6 and/or HC-HA. In some embodiments, the subject to be treated has high expression of TSG-6 and/or HC-HA in the lung. In some embodiments, the subject to be treated has increased airway eosinophilia.

In another aspect, the present invention relates to a method of treating an autoimmune or inflammatory disorder in a subject comprising administering to the subject an effective amount of an antibody, wherein the antibody serves as a TSG-6 agonist. In some embodiments, the autoimmune or inflammatory disorder is rheumatoid arthritis. In some embodiments, the subject to be treated has low expression of TSG-6 and/or HC-HA. In some embodiments, the antibody is combined with one or more additional therapeutic agents to treat an autoimmune or inflammatory disorder.

The present invention relates to an anti-TSG-6 antibody comprising: a heavy chain variable region comprising an amino acid sequence of SEQ ID NO:17 and a light chain variable region comprising an amino acid sequence of SEQ ID NO:18.

In some embodiments, the anti-TSG-6 antibody comprises: a vhCDR1 comprising SEQ ID NO:75, a vhCDR2 comprising SEQ ID NO:76, a vhCDR3 comprising SEQ ID NO:77, a vlCDR1 comprising SEQ ID NO:78, a vlCDR2 comprising SEQ ID NO:79, and a vlCDR3 comprising SEQ ID NO:80;

In some embodiments, the antibody binds human and/or mouse TSG-6.

In some embodiments, the anti-TSG-6 antibody comprises a constant region with an amino acid sequence at least 90% identical to a human IgG. In some embodiments, the human IgG is selected from a group consisting of IgG1, IgG2, IgG3 and IgG4. In some embodiments, the IgG is an IgG1.

In another aspect, the present invention relates to a nucleic acid composition encoding the anti-TSG-6 antibody described herein.

In another aspect, the present invention relates to a nucleic acid composition encoding the anti-TSG-6 antibody described herein. In some embodiments, the nucleic acid composition includes a first nucleic acid comprising the heavy chain, and a second nucleic acid comprising the light chain.

Another aspect of the present invention relates to an expression vector composition that includes any one of the nucleic acid compositions encoding the anti-TSG-6 antibody described herein. In some embodiments, the first nucleic acid is contained in a first expression vector and the second nucleic acid is contained in a second expression vector. In some other embodiments, the first nucleic acid and the second nucleic acid are contained in a single expression vector.

Another aspect of the present invention relates to a host cell that includes any one of the expression vectors described herein. Also presented is a method of making the anti-TSG-6 antibody, and the method includes culturing the host cell under conditions wherein the antibodies expressed, and recovering the antibodies.

In another aspect, the present invention relates to a composition that includes the anti-TSG-6 antibody described herein, and a pharmaceutical acceptable carrier or diluent.

In another aspect, the present invention relates to a method of modulating an immune response in a subject, and the method includes administering to the subject an effective amount of the anti-TSG-6 antibody described herein, or any one of the compositions described herein. In some embodiments, the method stimulates an immune response in the subject and the method includes administering to the subject an effective amount of an anti-TSG-6 antibody, or a composition thereof, that acts as a TSG-6 antagonist.

In another aspect, the present invention relates to a method of treating cancer in a subject comprising administering to the subject an effective amount of the anti-TSG-6 antibody, wherein the antibody serves as a TSG-6 antagonist. In some embodiments, the cancer has high expression of TSG-6 and/or HC-HA. In some embodiments, the subject to be treated has high expression of TSG-6 and/or HC-HA. In some embodiments, the cancer is a solid tumor. In some embodiments, the cancer is melanoma. In some embodiments, the antibody is combined with one or more additional therapeutic agents to treat cancer. In some embodiments, the additional therapeutic agents are other immune checkpoint inhibitors. In some embodiments, the other immune checkpoint inhibitors are selected from the group consisting of a PD-1 inhibitor, a PD-L1 inhibitor, a CTLA-4 inhibitor, a TIM-3 inhibitor, and a LAG-3 inhibitor.

In another aspect, the present invention relates to a method of treating fibrosis in a subject comprising administering to the subject an effective amount of the anti-TSG-6 antibody wherein the antibody serves as a TSG-6 antagonist. In some embodiments, the fibrotic tissue has high expression of TSG-6 and/or HC-HA. In some embodiments, the subject to be treated has high expression of TSG-6 and/or HC-HA. In some embodiments, the antibody is combined with one or more additional therapeutic agents to treat fibrosis.

In another aspect, the present invention relates to a method of treating an autoimmune or inflammatory disorder in a subject comprising administering to the subject an effective amount of an antibody, wherein the antibody serves as a TSG-6 antagonist. In some embodiments, the autoimmune or inflammatory disorder is idiopathic pulmonary artery hypertension. In some embodiments, the subject to be treated has high expression of TSG-6 and/or HC-HA. In some embodiments, the subject to be treated also has lung fibrosis. In some embodiments, the antibody is combined with one or more additional therapeutic agents to treat pulmonary artery hypertension and/or lung fibrosis. In some embodiments, the autoimmune or inflammatory disorder is asthma. In some embodiments, the subject to be treated has high expression of TSG-6 and/or HC-HA. In some embodiments, the subject to be treated has high expression of TSG-6 and/or HC-HA in the lung. In some embodiments, the subject to be treated has increased airway eosinophilia. In some embodiments, the antibody is combined with one or more additional therapeutic agents to treat an autoimmune or inflammatory disorder.

### BRIEF DESCRIPTION OF THE DRAWINGS

The invention may be best understood from the following detailed description when read in conjunction with the accompanying drawings. Included in the drawings are the following figures:

FIG. 1 shows inhibition of HA binding to TSG-6 by anti-TSG-6 antibodies.

FIG. 2 shows inhibition of TSG-6 HC-HA transesterase activity by anti-TSG-6 antibodies.

FIG. 3 shows pharmacodynamics of an anti-TSG-6 antibody.

FIGS. 4A to 4C show anti-tumor activity of the anti-TSG-6 antibody in tumors arising from B16F0 melanoma cells co-injected with CAFs.

FIGS. 5A to 5E show anti-tumor activity of the anti-TSG-6 antibody and its combination activity with anti-PD-1.

### DETAILED DESCRIPTION

The present disclosure provides novel anti-TSG-6 antibodies. The anti-TSG-6 antibodies described herein bind human and/or mouse TSG-6. In some embodiments, the anti-TSG-6 antibodies bind human and/or mouse TSG-6 with high affinities. In some embodiments, the anti-TSG-6 antibodies act as functional TSG-6 antagonists, and upon binding to TSG-6 they block interaction of TSG-6 with HA, and block HC-HA transesterase activity. In some embodiments, the anti-TSG-6 antibodies act as functional TSG-6 agonists, and upon binding to TSG-6 they increase interaction of TSG-6 with HA, and promote HC-HA transesterase activity. Also provided in the present disclosure are methods of using such antibodies to modulate an immune response in a subject, and, for example, to treat cancer, fibrosis, or an autoimmune or inflammatory disorder, including without limitation asthma.

To facilitate an understanding of the present invention, a number of terms and phrases are defined below.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

By “ablation” herein is meant a decrease or removal of activity. Thus for example, “ablating FcγR binding” means the Fc region amino acid variant has less than 50% starting binding as compared to an Fc region not containing the specific variant, with less than 70-80-90-95-98% loss of activity being preferred, and in general, with the activity being below the level of detectable binding in a Biacore assay.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the invention ablate ADCC activity entirely.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “antigen binding domain” or “ABD” herein is meant a set of six Complementary Determining Regions (CDRs) that, when present as part of a polypeptide sequence, specifically binds a target antigen as discussed herein. Thus, an “antigen binding domain” binds a target antigen as outlined herein. As is known in the art, these CDRs are generally present as a first set of variable heavy CDRs (vhCDRs or VHCDRs or CDR-HC) and a second set of variable light CDRs (vhCDRs or VLCDRs or CDR-LC), each comprising three CDRs: vhCDR1, vhCDR2, vhCDR3 for the heavy chain and vlCDR1, vlCDR2 and vlCDR3 for the light chain. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the VH and VL domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used. As is understood in the art, the CDRs are separated by framework regions in each of the variable heavy and variable light domains: for the light variable region, these are FR1-vlCDR1-FR2-vlCDR2-FR3-vlCDR3-FR4, and for the heavy variable region, these are FR1-vhCDR1-FR2-vhCDR2-FR3-vhCDR3-FR4, with the framework regions showing high identity to human germline sequences. Antigen binding domains of the invention include, Fab, Fv and scFv.

By “linker” herein is meant a linker used in scFv and/or other antibody structures. Generally, there are a number of suitable scFv linkers that can be used, including traditional peptide bonds, generated by recombinant techniques. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers. Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cy1, Cy2, Cy3, Cy4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins. In some embodiments, the linker is a “domain linker”, used to link any two domains as outlined herein together. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function.

The term “antibody” is used in the broadest sense and includes, for example, an intact immunoglobulin or an antigen binding portion. Antigen binding portions may be produced by recombinant DNA techniques or by enzymatic or chemical cleavage of intact antibodies. Thus the term antibody includes traditional tetrameric antibodies of two heavy chains and two light chains, as well as antigen binding fragments such as Fv, Fab and scFvs. In some cases, the invention provides bispecific antibodies that include at least one antigen binding domain as outlined herein.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M252Y refers to a variant polypeptide, in this case an Fc variant, in which the methionine at position 252 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95%-98%-99% identity. Variant protein can refer to the variant protein itself, compositions comprising the protein variant, or the DNA sequence that encodes it.

Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example M252Y or 252Y is an Fc variant with the substitution tyrosine at position 252 relative to the parent Fc polypeptide, wherein the numbering is according to the EU index. Likewise, M252Y/S254T/T256E defines an Fc variant with the substitutions M252Y, S254T and T256E relative to the parent Fc polypeptide. The identity of the wild type amino acid may be unspecified, in which case the aforementioned variant is referred to as 252Y/254T/256E. It is noted that the order in which substitutions are provided is arbitrary, that is to say that, for example, 252Y/254T/256E is the same Fc variant as 254T/252Y/256E, and so on. For all positions discussed in the present invention that relate to antibodies, unless otherwise noted, amino acid position numbering is according to Kabat for the variable region numbering and is according to the EU index for the constant regions, including the Fc region. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids.

As used herein, “protein” herein is meant at least two covalently attached amino acids, which includes proteins, polypeptides, oligopeptides and peptides. The peptidyl group may comprise naturally occurring amino acids and peptide bonds.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein.

By “Fv” or “Fv fragment” or “Fv region” as used herein is meant a polypeptide that comprises the VL and VH domains of a single antigen binding domain (ABD). As will be appreciated by those in the art, these generally are made up of two chains, or can be combined (generally with a linker as discussed herein) to form a scFv.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “heavy constant region” herein is meant the CH1-hinge-CH2-CH3 portion of an antibody, generally from human IgG1, IgG2 or IgG4.

By “target antigen” as used herein is meant the molecule that is bound specifically by the variable region of a given antibody. In the present case, the target antigen is a BTLA protein.

By “target cell” as used herein is meant a cell that expresses a target antigen.

By “variable region” as used herein is meant the region of an immunoglobulin that comprises one or more Ig domains substantially encoded by any of the V.kappa., V.lamda., and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

By “position” as used herein is meant a location in the sequence of a protein. Positions may be numbered sequentially, or according to an established format, for example the EU index for antibody numbering.

By “residue” as used herein is meant a position in a protein and its associated amino acid identity. For example, Asparagine 297 (also referred to as Asn297 or N297) is a residue at position 297 in the human antibody IgG1.

The antibodies of the present invention are generally recombinant. “Recombinant” means the antibodies are generated using recombinant nucleic acid techniques in exogenous host cells.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

The term “IC₅₀” or “half maximal inhibitory concentration”, as used herein, is intended to refer to the concentration of an inhibitor where the response (or binding) is reduced by half. IC₅₀ values for antibodies can be determined using methods well established in the art. In some embodiments, the method for determining the IC50 of an antibody is by using a 4-parameter logistic model.

A “disease” includes a state of health of an animal, including a human, wherein the animal cannot maintain homeostasis, and wherein if the disease is not ameliorated then the animal's health continues to deteriorate.

In contrast, a “disorder” in an animal, including a human, includes a state of health in which the animal is able to maintain homeostasis, but in which the animal's state of health is less favorable than it would be in the absence of the disorder. Left untreated, a disorder does not necessarily cause a further decrease in the animal's state of health. In some cases, “disorder” may be used interchangeably with “disease.”

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “mammal” refers to any mammal, including, but not limited to, mammals of the order Rodentia, such as mice and hamsters, and mammals of the order Logomorpha, such as rabbits. In some embodiments, the mammals are from the order Carnivora, including felines (cats) and canines (dogs). In some embodiments, the mammals are from the order Artiodactyla, including bovines (cows) and swines (pigs) or of the order Perssodactyla, including Equines (horses). It is most preferred that the mammals are of the order Primates, Ceboids, or Simoids (monkeys) or of the order Anthropoids (humans and apes). In some embodiments, the mammal is a human. In some embodiments, the mammal is cynomolgus monkey.

The term “regression,” as well as words stemming therefrom, as used herein, does not necessarily imply 100% or complete regression. Rather, there are varying degrees of regression of which one of ordinary skill in the art recognizes as having a potential benefit or therapeutic effect. In this respect, the disclosed methods can provide any amount of any level of regression of a cancer in a mammal. Furthermore, the regression provided by the inventive method can include regression of one or more conditions or symptoms of the disease, e.g., a cancer. Also, for purposes herein, “regression” can encompass delaying the onset of the disease, delaying the onset of a symptom, and/or delaying the onset of a condition thereof. With respect to progressive diseases and disorders, “regression” can encompass slowing the progression of the disease or disorder, slowing the progression of a symptom of the disease or disorder, and/or slowing the progression of a condition thereof.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

By “individual” or “host” or “subject” or “patient” is meant any mammalian subject for whom diagnosis, treatment, or therapy is desired, particularly humans. Other subjects may include cynomolgus monkey, cattle, dogs, cats, guinea pigs, rabbits, rats, mice, horses, and so on.

The term “in combination with” as used herein refers to uses where, for example, a first therapy is administered during the entire course of administration of a second therapy; where the first therapy is administered for a period of time that is overlapping with the administration of the second therapy, e.g., where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

“Encoding” includes the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if, for example, transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “nucleic acid” includes RNA or DNA molecules having more than one nucleotide in any form including single-stranded, double-stranded, oligonucleotide or polynucleotide. The term “nucleotide sequence” includes the ordering of nucleotides in an oligonucleotide or polynucleotide in a single-stranded form of nucleic acid.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes including non-native nucleic acid sequences, and the like.

The term “operably linked” as used herein includes a polynucleotide in functional relationship with a second polynucleotide, e.g., a single-stranded or double-stranded nucleic acid moiety comprising the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region. The order specified when indicating operably linkage is not important. For example, the phrases: “the promoter is operably linked to the nucleotide sequence” and “the nucleotide sequence is operably linked to the promoter” are used interchangeably herein and are considered equivalent. In some cases, when the nucleic acid encoding the desired protein further comprises a promoter/regulatory sequence, the promoter/regulatory sequence is positioned at the 5′ end of the desired protein coding sequence such that it drives expression of the desired protein in a cell.

The terms “oligonucleotide,” “polynucleotide,” and “nucleic acid molecule”, used interchangeably herein, refer to a polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

The term “recombinant,” as applied to a polynucleotide means the polynucleotide is the product of various combinations of cloning, restriction or ligation steps, and other procedures resulting in a construct distinct and/or different from a polynucleotide found in nature. The terms respectively include replicates of the original polynucleotide construct and progeny of the original virus construct.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors.

A “vector” is capable of transferring gene sequences to target-cells. Typically, “vector construct,” “expression vector,” and “gene transfer vector,” mean any nucleic acid construct capable of directing the expression of a gene of interest and which can transfer gene sequences to target-cells, which can be accomplished by genomic integration of all or a portion of the vector, or transient or inheritable maintenance of the vector as an extrachromosomal element. Thus, the term includes cloning, and expression vehicles, as well as integrating vectors.

The term “regulatory element” as used herein includes a nucleotide sequence which controls some aspect of the expression of nucleic acid sequences. Examples of regulatory elements illustratively include an enhancer, an internal ribosome entry site (IRES), an intron, an origin of replication, a polyadenylation signal (pA), a promoter, an enhancer, a transcription termination sequence, and an upstream regulatory domain, which contribute to the replication, transcription, and/or post-transcriptional processing of a nucleic acid sequence. In cases, regulatory elements can also include cis-regulatory DNA elements as well as transposable elements (TEs). Those of ordinary skill in the art are capable of selecting and using these and other regulatory elements in an expression construct with no more than routine experimentation. Expression constructs can be generated using a genetic recombinant approach or synthetically using well-known methodology.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

The statement that an amino acid residue is “phosphorylated” used herein means that a phosphate group is ester-linked to the side chain of the amino acid residue. Typical amino acid residues that may be phosphorylated include serine (Ser), threonine (Thr), and tyrosine (Tyr).

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various aspects of the invention are set forth below in sections; however, aspects of the invention described in one particular section are not to be limited to any particular section.

I. Antibodies

The present disclosure provides novel anti-TSG-6 antibodies. Such antibodies bind human and mouse TSG-6. Table 1 lists peptide sequences of heavy chain variable regions and light chain variable regions that, in combination as designated in Table 1, can bind to both human and mouse TSG-6. In some embodiments, the heavy chain variable region and the light chain variable region are arranged in a Fab format. In some embodiments, the heavy chain variable region and the light chain variable region are fused together to form an scFv.

TABLE 1

| | Heavy chain variable region | Light chain variable region |
|---|---|---|
| Chain | amino acid sequence | amino acid sequence |
| | MERHWIFLSLLSVIAGVHSQVRLQQSGAELAKPGASV | MDFQVQIFSFLLISASVIMSRGENVLTQSPAIMSASP |
| | KLSCKASGYTFTSYWMHWVKQRPGQGLEWIGYIIPSS | GEKVTMTCSASSSVSYMHWYQQKSSTSPKLWIYDTSK |
| | GYTKNKQNFKDKATLTADKSSSTAYMQLSNLTYEDSA | LASGVPGRFSGSGSGNSYSLTISSMEAEDVATYYCFQ |
| | VYSCARSDGSYPYYFDYWGQGTTLTVSSAKTTPPSVY | GSGYPLTFGSGTKLEIKRADAAPTVSIFPPSSEQLTS |
| | PLAPGSAAQTNSMVTLGCLVKGYFPEPVTVTWNSGSL | GGASVVCFLNNFYPRDINVKWKIDGSERQNGVLNSWT |
| | SSGVHTFPAVLQSDLYTLSSSVTVPSSTWPSQTVTCN | DQDSKDSTYSMSSTLTLTKDEYERHNSYTCEATHKTS |
| | VAHPASSTKVDKKIVPRDCGCKPCICTVPEVSSVFIF | TSP |
| | PPK | SEQ ID NO: 2 (kappa) |
| | SEQ ID NO: 1 (IgG1) | CDR1 (SEQ ID NO: 30)-SSVSY |
| | CDR1 (SEQ ID NO: 27)-GYTFTSYW | CDR2 (SEQ ID NO: 31)-DTS |
| | CDR2 (SEQ ID NO: 28)-IIPSSGYT | CDR3 (SEQ ID NO: 32)-FQGSGYPLT |
| | CDR3 (SEQ ID NO: 29)-ARSDGSYPYYFDY | |
| 2G11 | MGWSWIFLFLLSGTAGVHSQVQLQQSGPELVKPGASV | MKLPVRLLVLMFWIPASSSDVVMTQTPLSLPVSLGDQ |
| | KLSCKASGYTFTSYDINWVKQRPGQGLEWIGWIYPRD | ASISCRSSQSLVHSNGNTYLHWYLQKPGQSPKLLIYK |
| | GSTKYNEKFKDKATWTIDTSSNRAYMEIHSLTSEDSA | VSNRFSGVPDRFSGRGSGIDFTLKISRVEAEDLGVYF |
| | VYFCARGLWYYVSGMDYWGPGTSVTVSSAKTTPPSVY | CSQSTHVPPTFGGGTKLEIKRADAAPTVSIFPPSSEQ |
| | PLAPGSAAQTNSMVTLGCLVKGYFPEPVTVTWNSGLS | LTSGGASVVCFLNNFYPRDINVKWKIDGSERQNGVLN |
| | SGVHTFPAVLQSDLYTLSSSVTVPSSTWPSQTVTCNV | SWTDQDSKDSTYSMSSTLTLTKDEYERHNSYTCEATH |
| | AHPASSTKVDKKIVPRDCGCKPCICTVPEVSSVFIFP | KTSTSP |
| | PKPKDVLTITLTPKVTCVVVDISKDDQ | SEQ ID NO: 4 (kappa) |
| | SEQ ID NO: 3 (IgG1) | CDR1 (SEQ ID NO: 36)-QSLVHSNGNTY |
| | CDR1 (SEQ ID NO: 33)-GYTFTSYD | CDR2 (SEQ ID NO: 37)-KVS |
| | CDR2 (SEQ ID NO: 34)-IYPRDGST | CDR3 (SEQ ID NO: 38)-QSTHVPPT |
| | CDR3 (SEQ ID NO: 35)-ARGLWYYVSGMDY | |
| 3E1 | MERHWIFLSLLSVIAGVHSQVQLQQSGAELAKPGASV | MDFQVQIFSFLLISASVIMSRGENVLTQSPAIMSASP |
| | RLSCQASGYTFTSYWMHWVKQRPRQGLEWIGYIIPSS | GERVTMTCSATSSVSFMHWYQQKSSTSPKLWIYDTSK |
| | GYTKCHQKFKDKATLTADKSSSTAYMQLSSLTYEDSA | LASGVPGRFSGSGSGNSYSLTISSMEAEDVATYYCFQ |
| | VYYCARSTSGYPYYFDSWGQGTTLTVSSAKTTPPSVY | GSWYPLTFGAGTKLELKRADAAPTVSIFPPSSEQLTS |
| | PLAPGSAAQTNSMVTLGCLVKGYFPEPVTVTWNSGSL | GGASVVCFLNNFYPRDINVKWKIDGSERQNGVLNSWT |
| | SSGVHTFPAVLQSDLYTLSSSVTVPSSTWPSQTVTCN | DQDSKDSTYSMSSTLTLTKDEYERHNSYTCEATHKTS |
| | VAHPASSTKVDKKIVPRDCGCKPCICTVPEVSSVFIF | TSP |
| | PPKPKDVLTITLTPKVTCVVVDISKDDQ | SEQ ID NO: 6 (kappa) |
| | SEQ ID NO: 5 (IgG1) | CDR1 (SEQ ID NO: 42)-SSVSF |
| | CDR1 (SEQ ID NO: 39)-GYTFTSYW | CDR2 (SEQ ID NO: 43)-DTS |
| | CDR2 (SEQ ID NO: 40)-IIPSSGYT | CDR3 (SEQ ID NO: 44)-FQGSWYPLT |
| | CDR3 (SEQ ID NO: 41)-ARSTSGYPYYFDS | |
| 5A5 | MERHWIFLSLLSVIAGVHSQVQLQQSGAELAKPGASV | MDFQVQIFSFLLISASVIMSRGENVLTQSPAIMSASP |
| | RLSCQASGYTFTTYWMHWVKQRPRQGLEWIGYIIPSS | GEKVTMTCSATSSVSYMHWYQQKSSTSPKLWIYDTSK |
| | GYSKCHQKFKDKATLTADKSSNTASMQLSSLTYEDSA | LASGVPGRFSGSGSGNSYSLTISSMEAEDVATYYCFQ |
| | VYYCARSTSGYPYYFDYWGQGTTLTVSSAKTTPPSVY | GSWYPLTFGAGTKLELKRADAAPTVSIFPPSSEQLTS |
| | PLAPGSAAQTNSMVTLGCLVKGYFPEPVTVTWNSGLS | GGASVVCFLNNFYPRDINVKWKIDGSERQDGVLNSWT |
| | LSSGVHTFPAVLQSDLYTLSSSVTVPSSTWPSQTVTC | DQDSKDSTYSMSSTLTLTKDEYERHNSYTCEATHKTS |
| | NVAHPASSTKVDKKIVPRDCGCKPCICTVPEVSSVFI | TSP |
| | FPPKPKDVLTITLTPKVTCVVVDISKDDQ | SEQ ID NO: 8 (kappa) |
| | SEQ ID NO: 7 (IgG1) | CDR1 (SEQ ID NO: 48)-SSVSY |
| | CDR1 (SEQ ID NO: 45)-GYTFTTYW | CDR2 (SEQ ID NO: 49)-DTS |
| | CDR2 (SEQ ID NO: 46)-IIPSSGYS | CDR3 (SEQ ID NO: 50)-FQGSWYPLT |
| | CDR3 (SEQ ID NO: 47)-ARSTSGYPYYFD | |
| 4E4 | MGWSYIILFLVATATGVHSQVQLQQPGAELVKPGASV | MDFQVQIFSFLLISASVIMSRGQIVLTQSPAIMSASL |
| | KLSCKASGYTFTSYWMHWVKQRPGQGLEWIGMIHPNS | GERVTMTCTASSSVSSSYLHWYQQKPGSSPKLWIYST |
| | GSTNYNEKFKNKATLTVDKSSSTAYMQLSSLTSEDSA | SNLASGVPVRFSGSGSGTSYSLTISRMEAEDAATYYC |
| | VYYCAPRMFDDYDDYWGQGTTLTVSSAKTTPPSVYPL | HHYHRSPYTFGGGTKLEIKRADAAPTVSIFPPSSEQL |
| | APGSAAQTNSMVTLGCLVKGYFPEPVTVTWNSGSLSS | TSGGASVVCFLNNFYPRDINVKWKIDGSERQNGVLNS |
| | GVHTFPAVLQSDLYTLSSSVTVPSSTWPSQTVTCNVA | WTDQDSKDSTYSMSSTLTLTKDEYERHNSYTCEATHK |
| | HPASSTKVDKKIVPRDCGCKPCICTVPEVSSVFIFPP | TSTSP |
| | KPKDVLTITLTPKVTCVVVDISKDDQ | SEQ ID NO: 10 (kappa) |
| | SEQ ID NO: 9 (IgG1) | CDR1 (SEQ ID NO: 54)-SSVSSSY |
| | CDR1 (SEQ ID NO: 51)-GYTFTSYW | CDR2 (SEQ ID NO: 55)-STS |
| | CDR2 (SEQ ID NO: 52)-IHPNSGST | CDR3 (SEQ ID NO: 56)-HHYHRSPYT |
| | CDR3 (SEQ ID NO: 53)-APRMFDDYDDY | |
| 1A9 | MGWSWIFLFLLSGTAGVLSEVQLQQSGPELVKPGASV | MDMRTPAQFLGILLLWFPGIKCDIKMTQSPSSMYASL |
| | KISCKASGYTFTDYYMNWVKQSHEKSLEWIGDINPNN | GERVTIACKASQDINSFLSWFQQKPGKSPKTLIYRAN |
| | GGTSYNQKFMGKATLTVDKSSRTAYMELRSLTSEDTA | RLVDGVPSRFSGSGSGQDYSLTISSLEYEDMGIYYCL |
| | VYYCARWGIYDRFTYWGQGTLVSVSAAKTTPPSVYPL | QYDEFPLTFGAGTKLELKRADAAPTVSIFPPSSEQLT |
| | APGSAAQTNSMVTLGCLVKGYFPEPVTVTWNSGSLSS | SGGASVVCFLNNFYPRDINVKWKIDGSERQNGVLNSW |
| | GVHTFPAVLQSDLYTLSSSVTVPSSTWPSQTVTCNVA | TDQDSKDSTYSMSSTLTLTKDEYERHNSYTCEATHKT |
| | HPASSTKVDKKIVPRDCGCKPCICTVPEVSSVFIFPP | STSP |
| | KPKDVLTITLTPKCTCVVVDISKDDQG | SEQ ID NO: 12 (kappa) |
| | SEQ ID NO: 11 (IgG1) | CDR1 (SEQ ID NO: 60)-QDINSF |
| | CDR1 (SEQ ID NO: 57)-GYFTFDYY | CDR2 (SEQ ID NO: 61)-RAN |
| | CDR2 (SEQ ID NO: 58)-INPNNGGT | CDR3 (SEQ ID NO: 62)-LQYDEFPLT |
| | CDR3 (SEQ ID NO: 59)-ARWGIYDRFTY | |
| ID12 | MGWSWIFLFLLSGTAGVHSQVQLQQSGPELVKPGASV | MKLPVRLLVLMFWIPASSSDAVMTQIPLSLPVSLGDQ |
| | KLSCKTSGYTFTSYDINWVKQRPGQGLEWIGWIYPSD | ASISCRSTQSLEDSNGNTYLNWYLQKPGQSPQLLIYR |
| | GSTKFNEKFKGMATLTVDTSSSTAYMELHSLTSEDST | VSNRFSGVLDRFSGSGSGTDFTLKISRVEAEDLGVYF |
| | VYFCARGLWYYGGGVDYWGQGTSVTVSSAKTTPPSVY | CLQVTHVPYTFGGGTKLEIKRADAAPTVSIFPPSSEQ |
| | PLAPGSAAQTNSMVTLGCLVKGYFPEPVTVTWNSGSL | LTSGGASVVCFLNNFYPRDINVKWKIDGSERQNGVLN |
| | SSGVHTFPAVLQSDLYTLSSSVTVPSSTWPSQTVTCN | SWTDQDSKDSTYSMSSTLTLTKDEYERHNSYTCEATH |
| | VAHPASSTKVDKKIVPRDCGCKPCICTVPEVSSVFIF | KTSTSP |
| | PPKPKDVLTITLTPKVTCVVVDISKDDQ | SEQ ID NO: 14 (kappa) |
| | SEQ ID NO: 13 (IgG1) | CDR1 (SEQ ID NO: 66)-QSLEDSNGNTY |
| | CDR1 (SEQ ID NO: 63)-GYTFTSYD | CDR2 (SEQ ID NO: 67)-RVS |
| | CDR2 (SEQ ID NO: 64)-IYPSDGST | CDR3 (SEQ ID NO: 68)-LQVTHVPYT |
| | CDR3 (SEQ ID NO: 65)-ARGLWYYGGGVDY | |
| 1D7 | MGWSWIFLFLLSGTAGVLSEVQLQQSGPELVKPGASV | MDMRTPAQFLGILLLWFPGIKCDIKMTQSPSSMYASR |
| | KISCKASGYTFTDYYMNWVKQSHGKSLEWIGDINPNN | GERVTITCKASQDINSFLSWFQQKPGKSPKTLIYRAN |
| | GGTTYNQKFKGKATLTVDKSSSTAYMELRSLTSEDSA | RLVDGVPSRFSGSGSGQDYSLTISSLEYEDMGIYYCL |
| | VYYCARWGIFDRFTYWGQGTVVTVSAAKTTPPSVYPL | QYDEFPLTFGAGTKLELKRADAAPTVSIFPPSSEQLT |
| | APGSAAQTNSMVTLGCLVKGYFPEPVTVTWNSGSLSS | SGGASVVCFLNNFYPRDINVKWKIDGSERQNGVLNSW |
| | GVHTFPAVLQSDLYTLSSSVTVPSSTWPSQTVTCNVA | TDQDSKDSTYSMSSTLTLTKDEYERHNSYTCEATHKT |
| | HPASSTKVDKKIVPRDCGCKPCICTVPEVSSVFIFPP | STK |
| | KPKDVLTITLTPKVTCVVVDISKDDQ | SEQ ID NO: 16 |
| | SEQ ID NO: 15 (IgG1) | CDR1 (SEQ ID NO: 72)-QDINSF (kappa) |
| | CDR1 (SEQ ID NO: 69)-GYTFTDYY | CDR2 (SEQ ID NO: 73)-RAN |
| | CDR2 (SEQ ID NO: 70)-INPNNGGT | CDR3 (SEQ ID NO: 74)-LQYDEFPLT |
| | CDR3 (SEQ ID NO: 71)-ARWGIFDRFTY | |
| 3C6 | MGWSWIFLLSLPGTAGVLSEVQLQQSGPELVKPGASV | MDFQVQIFSFLLISASVIMSRGQIVLSQSPAILSASP |
| | KIPCKASGYTFTDYNMDWVKQSHGESLEWIGDIYPNN | GEKVTMTCRARSSVIYMHWYQQKPGSSPKPWIYATFN |
| | GGTIYNQKFKDKATLTVDKSSSTAYMELRSLTSEDNA | LASGVPARFSGSGSGTSYSLTISRVEAEDAATYYCQQ |
| | VYYCARKTGTGFDYWGQGTTLTVSSAKTTPPSVYPLA | WSSNPPTFGSGTKLEIKRADAAPTVSIFPPSSEQLTS |
| | PGSAAQTNSMVTLGCLVKGYFPEPVTVTWNSGSLSSG | GGASVVCFLNNFYPRDINVKWKIDGSERQNGVLNSWT |
| | VHTFPAVLQSDLYTLSSSVTVPSSTWPSQTVTCNVAH | DQDSKDSTYSMSSTLTLTKDEYERHNSYTCEATHKTS |
| | PASSTKVDKKIVPRDCGCKPCICTVPEVSSVFIFPPK | TSP |
| | SEQ ID NO: 17 (IgG1) | SEQ ID NO: 18 (kappa) |
| | CDR1 (SEQ ID NO: 75)-GYTFTDYN | CDR1 (SEQ ID NO: 78)-SVIY |
| | CDR2 (SEQ ID NO: 76)-IYPNNGGT | CDR2 (SEQ ID NO: 79)-ATF |
| | CDR3 (SEQ ID NO: 77)-ARKTGTGFDY | CDR3 (SEQ ID NO: 80)-QQWSSNPPT |
| 3E4 | MGWSYIILFLVATATGVHSQVQLQQPGAELVKPGASV | MKLPVRLLVLMFWIPASSTDVVMTQTPLSLPVSLGDL |
| | KLSCKASGYTFTSQWMHWVKQRPGQGLEWIGMIHPNS | ASISCRSSQSLVHSNGNTYLHWYLQKPGQSPKLLIFK |
| | GSTNNNEKFKSKATLTVDKSSSTAYMQLSSLTSEDSA | VSNRFSGVPDRFSGSGSGTDFTLKISRVEAEDLGVYF |
| | VYYCTRWAMDYWGQGTSVTASSAKTTPPSVYPLAPGS | CSQSTHVPWTFGGGTKLEIKRADAAPTVSIFPPSSEQ |
| | AAQTNSMVTLGCLVKGYFPEPVTVTWNSGSLSSGVHT | LTSGGASVVCFLNNFYPRDINVKWKIDGSERQNGVLN |
| | FPAVLQSDLYTLSSSVTVPSSTWPSQTVTCNVAHPAS | SWTDQDSKDSTYSMSSTLTLTKDEYERHNSYTCEATH |
| | STKVDKKIVPRDCGCKPCICTVPEVSSVFIFPPK | KTSTSTP |
| | SEQ ID NO: 19 (IgG1) | SEQ ID NO: 20 (kappa) |
| | CDR1 (SEQ ID NO: 81)-GYTFTSQW | CDR1 (SEQ ID NO: 84)-QSLVHSNGNTY |
| | CDR2 (SEQ ID NO: 82)-IHPNSGST | CDR2 (SEQ ID NO: 85)-KVS |
| | CDR3 (SEQ ID NO: 83)-TRWAMDY | CDR3 (SEQ ID NO: 86)-SQSTHVPWT |
| 4F1 | MKLWLNWIFLVTLLNGIQCEVKLVESGGGLVQPGGSL | MDFQVQIFSFLLISASVIISRGQIVLTQSPAIMSASP |
| | SLSCAASGFTFTDYYMSWVRQPPGKALEWLGFIRHKA | GEKVTMTCSASSSVSYMHWYQQKSGTSPKRWIYDTSK |
| | KGYTEAEYSASVKGRFTISRDNSQSILYLQMNALRAE | LASGVPARFSGSGSGTSYSLTISSMEAEDAATYYCQQ |
| | DSATYYCARLYYYGSPHWYFDVWGTGTTVTVSSAKTT | WSSNPPTFGSGTELEIKRADAAPTVSIFPPSSEQLTS |
| | PPSVYPLAPGSAAQTNSMVTLGCLVKGYFPEPVTVTW | GGASVVCFLNNFYPRDINVKWKIDGSERQNGVLNSWT |
| | NSGSLSSGVHTFPAVLQSDLYTLSSSVTVPSSTWPSQ | DQDSKDSTYSMSSTLTLTKDEYERHNSYTCEATHKTS |
| | TVTCNVAHPASSTKVDKKIVPRDCGCKPCICTVPEVS | TSP |
| | SVFIFPPKPKDVLTITLTPKVTCVVVDISKDDQ | SEQ ID NO: 22 (kappa) |
| | SEQ ID NO: 21 (IgG1) | CDR1 (SEQ ID NO: 90)-SSVSY |
| | CDR1 (SEQ ID NO: 87)-GFTFTDYY | CDR2 (SEQ ID NO: 91)-DTS |
| | CDR2 (SEQ ID NO: 88)-IRHKAKGYTA | CDR3 (SEQ ID NO: 92)-QQWSSNPPT |
| | CDR3 (SEQ ID NO: 89)-ARLYYYGSPHWYFDV | |
| 5B4 | MGWSCIMLFLAATATGVHSQVQLQQPGAELVKPGASV | MRCLAEFLGLLVLWIPGAIGDIVMTQAAPSVPVTPGE |
| | KLSCKASDYTFTNYWMHWVKQRPGRGLEWIGRIDPSS | SVSISCRSSKSLLHSNGNTYLYWFLQRPGQSPQLLIY |
| | GGAKYNEKFKSKATLTVDKPSSTAYMEFSSLTSEDSA | RMSNLASGVPDRFSGSGSGTAFTLRISRVEAEDVGVY |
| | VYYCVRSRYDYDGWFAYWGLGTLVTVSAAKTTPPSVY | YCMQHLEYPFTFGGGTKLEIKRADAAPTVSIFPPSSE |
| | PLAPGCGDTTGSSVTLGCLVKFYFPESVTVTWNSGSL | QLTSGGASVVCFLNNFYPRDINVKWKIDGSERQNGVL |
| | SSSVHTFPALLQSGLYTMSSSVTVPSSTWPSQTVTCS | NSWTDQDSKDSTYSMSSTLTLTKDEYERHNSYTCEAT |
| | VAHPASSTTVDKKLEPSGPISTINPCPPCKECHKCPA | HKTSTSP |
| | PNLEGGPSVFIFPPNIKDVLMISLTPKVTCVVVDVSE | SEQ ID NO: 24 (kappa) |
| | DDQ | CDR1 (SEQ ID NO: 96)-KSLLHSNGNTYLY |
| | SEQ ID NO: 23 (IgG1) | CDR2 (SEQ ID NO: 97)-RMS |
| | CDR1 (SEQ ID NO: 93)-DYTFTNYW | CDR3 (SEQ ID NO: 98)-MQHLEYPFT |
| | CDR2 (SEQ ID NO: 94)-IDPSSGGA | |
| | CDR3 (SEQ ID NO: 95)-VRSRYDYDGWFAY | |
| 5D9 | MEWIWIFLFILSGTAGVQSQVQLQQSGAELARPGASV | MESQTQVLISLLFWVSGTCGDIVMTQSPSSLSVSAGE |
| | KLSCKASDDTFINYGINWVKQRTGQGLEWIGETFPSN | KVTMSCKSSQSLLNSGNQKNYLAWYQQKPGQPPKLLI |
| | GNTFYNEKFKGKATLTADRSSSTTYMELRSLTSEDAA | YGASTRESGVPERFTGSGSGTDFTLTISSVQAEDLAV |
| | VYFCARHSNLPYFDHWGQGSTLTVSSAKTTPPSVYPL | YYCQNDHSYPFTFGGGTNLEIKRADAAPTVSIFPPSS |
| | APGSAAQTNSMVTLGCLVKGYFPEPVTVTWNSGSLSS | EQLTSGGASVVCFLNNFYPRDINVKWKIDGSERQNGV |
| | GVHTFPAVLQSDLYTLSSSVTVPSSTWPSQTVTCNVA | LNSWTDQDSKDSTYSMSSTLTLTKDEYERHNSYTCEA |
| | HPASSTKVDKKIVPRDCGCKPCICTVPEVSSVFIFPP | THKTSTSP |
| | KPKDVLTITLTPKVTCVVVDISKDDQ | SEQ ID NO: 26 (kappa) |
| | SEQ ID NO: 25 (IgG1) | CDR1 (SEQ ID NO: 102)-QSLLNSGNQKNY |
| | CDR1 (SEQ ID NO: 99)-DDTFINYG | CDR2 (SEQ ID NO: 103)-GAS |
| | CDR2 (SEQ ID NO: 100)-TFPSNGNT | CDR3 (SEQ ID NO: 104)-QNDHSYPFT |
| | CDR3 (SEQ ID NO: 101)-ARHSNLPYFDH | |

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:1 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:2.

In some embodiments, the anti-TSG-6 antibodies include a vhCDR1 comprising SEQ ID NO:27, a vhCDR2 comprising SEQ ID NO:28, a vhCDR3 comprising SEQ ID NO:29, a vlCDR1 comprising SEQ ID NO:30, a vlCDR2 comprising SEQ ID NO:31, and a vlCDR3 comprising SEQ ID NO:32. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:3 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:4.

In some embodiments, the anti-TSG-6 antibodies include a vhCDR1 comprising SEQ ID NO:33, a vhCDR2 comprising SEQ ID NO:34, a vhCDR3 comprising SEQ ID NO:35, a vlCDR1 comprising SEQ ID NO:36, a vlCDR2 comprising SEQ ID NO:37, and a vlCDR3 comprising SEQ ID NO:38. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:5 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:6.

In some embodiments, the anti-TSG-6 antibodies include a vhCDR1 comprising SEQ ID NO:39, a vhCDR2 comprising SEQ ID NO:40, a vhCDR3 comprising SEQ ID NO:41, a vlCDR1 comprising SEQ ID NO:42, a vlCDR2 comprising SEQ ID NO:43, and a vlCDR3 comprising SEQ ID NO:44. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:7 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:8.

In some embodiments, the anti-TSG-6 antibodies include a vhCDR1 comprising SEQ ID NO:45, a vhCDR2 comprising SEQ ID NO:46, a vhCDR3 comprising SEQ ID NO:47, a vlCDR1 comprising SEQ ID NO:48, a vlCDR2 comprising SEQ ID NO:49, and a vlCDR3 comprising SEQ ID NO:50. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:9 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:10.

In some embodiments, the anti-TSG-6 antibodies include a vhCDR1 comprising SEQ ID NO:51, a vhCDR2 comprising SEQ ID NO:52, a vhCDR3 comprising SEQ ID NO:53, a vlCDR1 comprising SEQ ID NO:54, a vlCDR2 comprising SEQ ID NO:55, and a vlCDR3 comprising SEQ ID NO:56. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:11 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:12.

In some embodiments, the anti-TSG-6 antibodies that include a vhCDR1 comprising SEQ ID NO:57, a vhCDR2 comprising SEQ ID NO:58, a vhCDR3 comprising SEQ ID NO:59, a vlCDR1 comprising SEQ ID NO:60, a vlCDR2 comprising SEQ ID NO:61, and a vlCDR3 comprising SEQ ID NO:62. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:13 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:14.

In some embodiments, the anti-TSG-6 antibodies that include a vhCDR1 comprising SEQ ID NO:63, a vhCDR2 comprising SEQ ID NO:64, a vhCDR3 comprising SEQ ID NO:65, a vlCDR1 comprising SEQ ID NO:66, a vlCDR2 comprising SEQ ID NO:67, and a vlCDR3 comprising SEQ ID NO:68. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:15 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:16.

In some embodiments, the anti-TSG-6 antibodies that include a vhCDR1 comprising SEQ ID NO:69, a vhCDR2 comprising SEQ ID NO:70, a vhCDR3 comprising SEQ ID NO:71, a vlCDR1 comprising SEQ ID NO:72, a vlCDR2 comprising SEQ ID NO:73, and a vlCDR3 comprising SEQ ID NO:74. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:17 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:18.

In some embodiments, the anti-TSG-6 antibodies include a vhCDR1 comprising SEQ ID NO:75, a vhCDR2 comprising SEQ ID NO:76, a vhCDR3 comprising SEQ ID NO:77, a vlCDR1 comprising SEQ ID NO:78, a vlCDR2 comprising SEQ ID NO:79, and a vlCDR3 comprising SEQ ID NO:80. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:19 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:20.

In some embodiments, the anti-TSG-6 antibodies include a vhCDR1 comprising SEQ ID NO:81, a vhCDR2 comprising SEQ ID NO:82, a vhCDR3 comprising SEQ ID NO:83, a vlCDR1 comprising SEQ ID NO:84, a vlCDR2 comprising SEQ ID NO:85, and a vlCDR3 comprising SEQ ID NO:86. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:21 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:22.

In some embodiments, the anti-TSG-6 antibodies include a vhCDR1 comprising SEQ ID NO:87, a vhCDR2 comprising SEQ ID NO:88, a vhCDR3 comprising SEQ ID NO:89, a vlCDR1 comprising SEQ ID NO:90, a vlCDR2 comprising SEQ ID NO:91, and a vlCDR3 comprising SEQ ID NO:92. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:23 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:24.

In some embodiments, the anti-TSG-6 antibodies include a vhCDR1 comprising SEQ ID NO:93, a vhCDR2 comprising SEQ ID NO:94, a vhCDR3 comprising SEQ ID NO:95, a vlCDR1 comprising SEQ ID NO:96, a vlCDR2 comprising SEQ ID NO:97, and a vlCDR3 comprising SEQ ID NO:98. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the anti-TSG-6 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:25 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:26.

In some embodiments, the anti-TSG-6 antibodies include a vhCDR1 comprising SEQ ID NO:99, a vhCDR2 comprising SEQ ID NO:100, a vhCDR3 comprising SEQ ID NO:101, a vlCDR1 comprising SEQ ID NO:102, a vlCDR2 comprising SEQ ID NO:103, and a vlCDR3 comprising SEQ ID NO:104. In some embodiments, one or more of such 6 CDRs have from 1, 2, 3, 4 or 5 amino acid modifications. In further embodiments, a single CDR contains 1 or 2 amino acid substitutions, and the modified anti-TSG-6 antibodies retain binding to human and/or mouse TSG-6.

In some embodiments, the present disclosure includes a nucleic acid encoding any one of the amino acid sequence variants described herein. In some embodiments, the present disclosure includes an expression vector comprising any of the nucleic acids described herein. In some embodiments, the present disclosure includes a host cell comprising any one of the expression vectors described herein.

In addition to the sequence variants described herein in the heavy chain and light chain variable regions and/or CDRs, changes in the framework region(s) of the heavy and/or light variable region(s) can be made. In some embodiments, variants in the framework regions (e.g., excluding the CDRs) retain at least about 80, 85, 90 or 95% identity to a germline sequence. Variants can be made to retain at least about 80, 85, 90 or 95% identity to any one of the light chain V-GENE, light chain J-GENE, heavy chain V-GENE, heavy chain J-GENE, and heavy chain D-GENE alleles.

In some embodiments, variations are made in the framework regions that retain at least 80, 85, 90 or 95% identity to the germline gene sequences, while keeping 6 CDRs unchanged.

In some embodiments, variations are made in both the framework regions that retain at least 80, 85, 90 or 95% identity to the germline gene sequences, and the 6 CDRs. The CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.).

By selecting amino acid sequences of CDRs and/or variable regions of a heavy chain and a light chain from those described herein and combining them with amino acid sequences of framework regions and/or constant regions of a heavy chain and a light chain of an antibody as appropriate, a person skilled in the art will be able to design an anti-TSG-6 antibody according to the present invention. The antibody framework regions and/or constant region (Fc domain) described in the current invention can derive from an antibody of any species, such as from human, rabbit, dog, cat, mouse, horse or monkey.

In some embodiments, the constant region is derived from human, and includes a heavy chain constant region derived from those of IgG, IgA, IgM, IgE, and IgD subtypes or variants thereof, and a light chain constant region derived from kappa or lambda subtypes or variants thereof. In some embodiments, the heavy chain constant region is derived from a human IgG, including IgG1, IgG2, IgG3, and IgG4. In some embodiments, the amino acid sequence of the heavy chain constant region is at least 80%, 85%, 90%, or 95% identical to a human IgG1, IgG2, IgG3, or IgG4 constant region. In some other embodiments, the amino acid sequence of the constant region is at least 80%, 85%, 90%, or 95% identical to an antibody constant region from another mammal, such as rabbit, dog, cat, mouse, horse or monkey. In some embodiments, the antibody constant region includes a hinge, a CH2 domain, a CH3 domain and optionally a CH1 domain.

In some embodiments, the antibodies described herein can be derived from a mixture from different species, e.g., forming a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system, as described for example in Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci, USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. Other humanization methods may involve the grafting of only parts of the CDRs, including but not limited to methods described in Tan et al., 2002, J. Immunol. 169:1119-1125; De Pascalis et al., 2002, J. Immunol. 169:3076-3084, all entirely incorporated by reference.

In some embodiments, the antibodies of the current invention comprise a heavy chain variable region derived from a particular human germline heavy chain immunoglobulin gene and/or a light chain variable region derived from a particular human germline light chain immunoglobulin gene. Such antibodies may contain amino acid differences as compared to the human germline sequences, due to, for example, naturally-occurring somatic mutations or intentional introduction of site-directed mutation. However, a humanized antibody typically is at least 80% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the human germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In some embodiments, the antibodies of the current disclosure are humanized and affinity matured, as is known in the art. Structure-based methods may be employed for humanization and affinity maturation, for example as described in U.S. Pat. No. 7,657,380. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference.

II. Characteristics of the Antibodies

In some embodiments, the anti-TSG-6 antibodies described herein bind to human and/or mouse TSG-6. In some embodiments, binding of the anti-TSG-6 antibodies to human and/or mouse TSG-6 is measured by studying binding to HA using ELISA, such as the exemplary assay described in Example 2. In such embodiments, antibodies described herein display an IC50 that can range from 10-2000 nM as measured by such assays. In some embodiments, binding of the anti-TSG-6 antibodies to human and/or mouse TSG-6 is measured by studying TSG-6 HC-HA transesterase activity using ELISA, such as the exemplary assay described in Example 2. In such embodiments, the antibodies described herein display an IC50 that ranges from 4-3000 nM as measured by ELISA. In further embodiments, the IC50 of antibodies described herein range from about 0.1-4000, 0.1-3000, 1-2800, 2-2600, 3-2400, 4-2200, 5-2000, 6-1800, 7-1600, 8-1400, or 9-1200 nM as measured by ELISA. In some embodiments, the IC50 of antibodies described herein range from 50-3500, 100-3000, 150-2500, 200-2000, 250-1500, and 300-1000 nM as measured by ELISA.

In some embodiments, anti-TSG-6 antibodies described act as TSG-6 antagonists, and block interaction of TSG-6 with HA as well as production of HC-HA. As a result, such anti-TSG-6 antibodies stimulate an immune response.

In some embodiments, the anti-TSG-6 antibodies described herein reduce levels of HC-HA. In some embodiments, the reduction in HC-HA is measured using ELISA, such as in the exemplary assay described in Example 3.

In some embodiments, the anti-TSG-6 antibodies described herein reduce tumor volume, such as in the exemplary assay described in Example 4.

In some other embodiments, anti-TSG-6 antibodies described herein act as TSG-6 agonists, and suppress immune cell functions, including pro-inflammatory T cell functions. As a result, such anti-TSG-6 antibodies suppress an immune response.

In other embodiments, the anti-TSG-6 antibodies described herein increase levels of HC-HA. In some embodiments, the increase in HC-HA is measured using ELISA, such as in the exemplary assay described in Example 3.

III. Nucleic Acids of the Invention

Nucleic acids encoding the anti-TSG-6 antibodies also fall within the scope of the present invention, as well as expression vectors containing such nucleic acids and host cells transformed with such nucleic acids and/or expression vectors. As will be appreciated by those in the art, the protein sequences can be encoded by any number of possible nucleic acid sequences due to the degeneracy of the genetic code.

Nucleic acid compositions encoding the anti-TSG-6 antibodies and/or TSG-6-binding domains also fall within the scope of the present invention. As will be appreciated by those in the art, in the case of antigen binding domains, the nucleic acid compositions generally include a first nucleic acid encoding the heavy chain variable region and a second nucleic acid encoding the light chain variable region. In the case of scFvs, a single nucleic acid encoding the heavy chain variable region and light chain variable region, separated by a linker described herein, can be made. In the case of traditional antibodies, the nucleic acid compositions generally include a first nucleic acid encoding the heavy chain and a second nucleic acid encoding the light chain, which will, upon expression in a cell, spontaneously assemble into the “traditional” tetrameric format of two heavy chains and two light chains.

As is known in the art, the nucleic acids encoding the components of the invention can be incorporated into expression vectors, and depending on the host cells, used to produce the antibodies of the invention. These two nucleic acids can be incorporated into a single expression vector or into two different expression vectors. Generally, the nucleic acids can be operably linked to any number of regulatory elements (promoters, origin of replication, selectable markers, ribosomal binding sites, inducers, etc.) in an expression vector. The expression vectors can be extra-chromosomal or integrating vectors.

The nucleic acids and/or expression vectors of the current invention can be introduced into any type of host cells, which are well known in the art, including mammalian, bacterial, yeast, insect and fungal cells. After transfection, single cell clones can be isolated for cell bank generation using methods known in the art, such as limited dilution, ELISA, FACS, microscopy, or Clonepix. Clones can be cultured under conditions suitable for bio-reactor scale-up and maintained expression of the antibodies. The antibodies can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

IV. Therapeutic Applications

The current disclosure provides a method of modulating an immune response in a subject, and the method includes administering to the subject an effective amount of an anti-TSG-6 antibody described herein, or a pharmaceutical composition containing an anti-TSG-6 antibody.

In some embodiments, the methods of modulating an immune response encompassed by the present disclosure comprises stimulating an immune response in a subject, and in further embodiments, such methods comprise administering to the subject an effective amount of an anti-TSG-6 antibody that acts as a TSG-6 antagonist, or by administering a pharmaceutical composition containing an antagonistic anti-TSG-6 antibody.

In some embodiments, the present disclosure provides methods for suppressing an immune response in a subject, for example, by administering to the subject an effective amount of an anti-TSG-6 antibody that acts as a TSG-6 agonist, or by administering to the subject a pharmaceutical composition containing such an agonistic anti-TSG-6 antibody.

The present disclosure also provides methods of treating cancer in a subject, and such methods include administering to the subject an effective amount of an anti-TSG-6 antibody that acts as a TSG-6 antagonist, or a pharmaceutical composition containing such anti-TSG-6 antibody. In some embodiments, the cancer to be treated has high expression of HC-HA and/or TSG-6 compared to the corresponding non-cancerous tissue. In some embodiments, the cancer to be treated uses the TSG-6/HA pathway to promote cancer progression. In some embodiments, the cancer to be treated uses TSG-6 to catalyze the transfer of HC proteins from inter-alpha-inhibitor (IαI) to HA in a transesterification reaction, forming an HC-HA complex. In some embodiments, the HC-HA complex forms cable-like structures which have altered binding properties, alter the polarization of macrophages to the ‘M2’ phenotype, and increase the pathogenesis of cancer. In some embodiments, the cancer to be treated is non-responsive to existing immune-modulating antibodies targeting other immune checkpoints, such as CTLA-4, PD-1 or PD-L1.

In some embodiments, the cancer is a solid tumor, such as gastric cancer, colorectal cancer, hepatocellular carcinoma, melanoma, or esophageal squamous cell carcinoma. In some embodiments, the cancer is B-cell chronic lymphocytic leukemia, Hodgkin's lymphoma, B-cell non-Hodgkin's lymphoma or T-cell non-Hodgkin's lymphomas.

In some other embodiments, the cancer is brain cancer, bladder cancer, breast cancer, cervical cancer, endometrial cancer, esophageal cancer, leukemia, lung cancer, liver cancer, melanoma, ovarian cancer, pancreatic cancer, prostate cancer, rectal cancer, renal cancer, testicular cancer, or uterine cancer. In yet other embodiments, the cancer is a vascularized tumor, squamous cell carcinoma, adenocarcinoma, small cell carcinoma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well-differentiated carcinoma, or Wilms tumor.

In some other embodiments, the cancer to be treated is a non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The present disclosure also provides methods of treating fibrosis in a subject, and the method includes administering to the subject an effective amount of an anti-TSG-6 antibody that acts as a TSG-6 antagonist, or a pharmaceutical composition containing such anti-TSG-6 antibody. In some embodiments, the fibrotic tissue has high expression of TSG-6 and/or HC-HA. In some embodiments, the subject has high expression of TSG-6 and/or HC-HA. In some embodiments, the HC-HA complex forms cable-like structures which have altered binding properties, alter the polarization of macrophages to the ‘M2’ phenotype, and increase the pathogenesis of fibrosis. In some embodiments, the antibody is combined with one or more additional therapeutic agents to treat fibrosis.

In some embodiments, fibrosis includes, but is limited to, collagen disease, interstitial lung disease, human fibrotic lung disease (e.g., obliterative bronchiolitis, idiopathic pulmonary fibrosis, pulmonary fibrosis from a known etiology, tumor stroma in lung disease, systemic sclerosis affecting the lungs, Hermansky-Pudlak syndrome, coal worker's pneumoconiosis, asbestosis, silicosis, chronic pulmonary hypertension, AIDS-associated pulmonary hypertension, sarcoidosis, and the like), fibrotic vascular disease, arterial sclerosis, atherosclerosis, varicose veins, coronary infarcts, cerebral infarcts, myocardial fibrosis, musculoskeletal fibrosis, post-surgical adhesions, human kidney disease (e.g., nephritic syndrome, Alport's syndrome, HIV-associated nephropathy, polycystic kidney disease, Fabry's disease, diabetic nephropathy, chronic glomerulonephritis, nephritis associated with systemic lupus, and the like), cutis keloid formation, progressive systemic sclerosis (PSS), primary sclerosing cholangitis (PSC), liver fibrosis, liver cirrhosis, renal fibrosis, pulmonary fibrosis, cystic fibrosis, chronic graft versus host disease, scleroderma (local and systemic), Grave's opthalmopathy, diabetic retinopathy, glaucoma, Peyronie's disease, penis fibrosis, urethrostenosis after the test using a cystoscope, inner accretion after surgery, scarring, myelofibrosis, idiopathic retroperitoneal fibrosis, peritoneal fibrosis from a known etiology, drug-induced ergotism, fibrosis incident to benign or malignant cancer, fibrosis incident to microbial infection (e.g., viral, bacterial, parasitic, fungal, etc.), Alzheimer's disease, fibrosis incident to inflammatory bowel disease (including stricture formation in Crohn's disease and microscopic colitis), fibrosis induced by chemical or environmental insult (e.g., cancer chemotherapy, pesticides, radiation (e.g., cancer radiotherapy), and the like), and the like. In some embodiments, “fibrotic tissue” is any tissue that is affected by but not limited to fibrosis that comprises one of these examples.

The present disclosure also provides methods of treating an autoimmune or inflammatory disorder in a subject, and the method includes administering to the subject an effective amount of an anti-TSG-6 antibody or a pharmaceutical composition containing such anti-TSG-6 antibody. In some embodiments, the TSG-6 antibody is an antagonist. In other embodiments, the TSG-6 antibody is an agonist.

In one aspect, methods of treating an autoimmune or inflammatory disorder in a subject include administering to the subject an effective amount of an anti-TSG-6 antibody that acts as a TSG-6 antagonist, or a pharmaceutical composition containing such anti-TSG-6 antibody. In some embodiments, the subject to be treated has high expression of TSG-6 and/or HC-HA. In some embodiments, the subject to be treated has high expression of TSG-6 and/or HC-HA in an inflamed tissue. Administering an anti-TSG-6 antibody that acts as a TSG-6 antagonist can stimulate an immune response to resolve inflammation that occurs as a result of an autoimmune or inflammatory disorder.

In some embodiments, the autoimmune or inflammatory disorder to be treated with an antagonistic anti-TSG-6 antibody is asthma. In some embodiments, the subject to be treated has high expression of TSG-6 and/or HC-HA in the lung. In some embodiments, the subject to be treated has increased airway eosinophilia. In some embodiments the antibody is combined with one or more additional therapeutics to treat asthma.

In some embodiments, the autoimmune or inflammatory disorder to be treated with an antagonistic anti-TSG-6 antibody is idiopathic pulmonary artery hypertension. In some embodiments, the subject has high expression of TSG-6 and/or HC-HA. In some embodiments the subject to be treated also has lung fibrosis. In some embodiments, the antibody is combined with additional therapeutic agents to treat idiopathic pulmonary artery hypertension and/or lung fibrosis.

In another aspect, methods of treating an autoimmune or inflammatory disorder in a subject include administering to the subject an effective amount of an anti-TSG-6 antibody that acts as a TSG-6 agonist, or a pharmaceutical composition containing such anti-TSG-6 antibody. In some embodiments, the subject to be treated has low expression of TSG-6 and/or HC-HA. In some embodiments, the subject to be treated has low expression of TSG-6 and/or HC-HA in an inflamed tissue. Administering an anti-TSG-6 antibody that acts as a TSG-6 agonist can suppress a pro-inflammatory immune response, including pro-inflammatory T cell response, and modulate immune responses in the subject suffering from an autoimmune or inflammatory disorder.

In some embodiments, the autoimmune or inflammatory disorder to treated with an agonistic anti-TSG-6 antibody is rheumatoid arthritis. Administering an anti-TSG-6 antibody that acts as a TSG-6 agonist can suppress a pro-inflammatory immune response by inhibiting migration of pathogenic cells.

In some embodiments, the autoimmune or inflammatory disorder to be treated with an antagonistic or agonistic anti-TSG-6 antibody is atherosclerosis, multiple sclerosis, Addison's disease, amyotrophic lateral sclerosis, Crohn's disease, Cushing's Syndrome, diabetes mellitus type 1, graft versus host disease, Graves' disease, Guillain-Barré syndrome, inflammatory bowel disease, lupus erythematosus, psoriasis, psoriatic arthritis, rheumatoid arthritis, sarcoidosis, scleroderma, systemic lupus erythematosus, transplant rejection, or vasculitis.

In some embodiments, the autoimmune or inflammatory disorder to be treated with an antagonistic or agonistic anti-TSG-6 antibody is an inflammatory disorder of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, spinal cord, testis, thymus, thyroid or uterus.

In some other embodiments, the autoimmune or inflammatory disorders to be treated with an antagonistic or agonistic anti-TSG-6 antibody include, but are not limited to, Acute disseminated encephalomyelitis (ADEM), Agammaglobulinemia, Alopecia areata, Ankylosing Spondylitis, Antiphospholipid syndrome, Antisynthetase syndrome, Atopic allergy, Atopic dermatitis, Autoimmune aplastic anemia, Autoimmune cardiomyopathy, Autoimmune enteropathy, Autoimmune hemolytic anemia, Autoimmune hepatitis, Autoimmune inner ear disease, Autoimmune lymphoproliferative syndrome, Autoimmune pancreatitis, Autoimmune peripheral neuropathy, Autoimmune polyendocrine syndrome, Autoimmune progesterone dermatitis, Autoimmune thrombocytopenic purpura, Autoimmune urticaria, Autoimmune uveitis, Balo disease/Balo concentric sclerosis, Behcet's disease, Berger's disease, Bickerstaffs encephalitis, Blau syndrome, Bullous pemphigoid, Bronchopulmonary dysplasia, Cancer, Castleman's disease, Celiac disease, Chagas disease, Chronic inflammatory demyelinating polyneuropathy, Chronic inflammatory demyelinating polyneuropathy, Chronic obstructive pulmonary disease, Chronic recurrent multifocal osteomyelitis, Churg-Strauss syndrome, Cicatricial pemphigoid, Cogan syndrome, Cold agglutinin disease, Complement component 2 deficiency, Contact dermatitis, Cranial arteritis, CREST syndrome, Cutaneous leukocytoclastic angiitis, Dego's disease, Dercum's disease, Dermatitis herpetiformis, Dermatomyositis, Diffuse cutaneous systemic sclerosis, Discoid lupus erythematosus, Dressler's syndrome, Drug-induced lupus, Eczema, Endometriosis, Eosinophilic fasciitis, Eosinophilic gastroenteritis, Eosinophilic pneumonia, Epidermolysis bullosa acquisita, Erythema nodosum, Erythroblastosis fetalis, Essential mixed cryoglobulinemia, Evan's syndrome, Fibrodysplasia ossificans progressiva, Fibrosing alveolitis (or Idiopathic pulmonary fibrosis), Gastritis, Gastrointestinal pemphigoid, Glomerulonephritis, Goodpasture's syndrome, Hashimoto's encephalopathy, Hashimoto's thyroiditis, Henoch-Schonlein purpura, Herpes gestationis aka Gestational Pemphigoid, Hidradenitis suppurativa, Hughes-Stovin syndrome, Hypogammaglobulinemi, Idiopathic inflammatory demyelinating diseases, Idiopathic pulmonary fibrosis, Idiopathic thrombocytopenic purpura, IgA nephropathy, Inclusion body myositis, Interstitial cystitis, Juvenile idiopathic arthritis aka Juvenile rheumatoid arthritis, Kawasaki's disease, Lambert-Eaton myasthenic syndrome, Leukocytoclastic vasculitis, Lichen planus, Lichen sclerosus, Linear IgA disease, Lupoid hepatitis aka Autoimmune hepatitis, Majeed syndrome, Microscopic colitis, Microscopic polyangiitis, Miller-Fisher syndrome, Mixed connective tissue disease, Morphea, Mucha-Habermann disease aka Pityriasis lichenoides et varioliformis acuta, Multiple sclerosis, Myasthenia gravis, Myositis, Meniere's disease, Narcolepsy, Neuromyelitis optica, Neuromyotonia, Occular cicatricial pemphigoid, Opsoclonus myoclonus syndrome, Ord's thyroiditis, Palindromic rheumatism, PANDAS (pediatric autoimmune neuropsychiatric disorders associated with streptococcus), Paraneoplastic cerebellar degeneration, Paroxysmal nocturnal hemoglobinuria (PNH), Parry Romberg syndrome, Pars planitis, Parsonage-Turner syndrome, Pemphigus vulgaris, Perivenous encephalomyelitis, Pernicious anaemia, POEMS syndrome, Polyarteritis nodosa, Polymyalgia rheumatica, Polymyositis, Primary biliary cirrhosis, Primary sclerosing cholangitis, Progressive inflammatory neuropathy, Pure red cell aplasia, Pyoderma gangrenosum, Rasmussen's encephalitis, Raynaud phenomenon, Reiter's syndrome, Relapsing polychondritis, Restless leg syndrome, Retroperitoneal fibrosis, Rheumatic fever, Schizophrenia, Schmidt syndrome, Schnitzler syndrome, Scleritis, Serum Sickness, Sjögren's syndrome, Spondyloarthropathy, Stiff person syndrome, Still's disease, Subacute bacterial endocarditis (SBE), Susac's syndrome, Sweet's syndrome, Sydenham chorea, Sympathetic ophthalmia, Takayasu's arteritis, Temporal arteritis, Thrombocytopenia, Tolosa-Hunt syndrome, Transverse myelitis, Ulcerative colitis, Undifferentiated spondyloarthropathy, Urticarial vasculitis, Vitiligo, Wegener's granulomatosis.

V. Combination Therapy

Anti-TSG-6 antibodies described herein can be used in combination with additional therapeutic agents to treat cancer, fibrosis, or autoimmune or inflammatory disorders.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

In certain aspects, the expression of one or more proteins involved in the immune response is inhibited as part of a combination therapy in treating cancer. Agents that act as “inhibitors” may bind directly to the protein, for example antibodies, antibody-drug conjugates, soluble proteins, and fusion proteins. In some embodiments, inhibitors used in combination therapy are, for example, oligonucleotides, siRNA, miRNA, piRNA, and shRNA. In some embodiments, inhibitors used in combination therapy block proteasome function. In some embodiments, inhibitors used in combination therapy block DNA methylation.

Among inhibitors of use in the present invention are immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3, such as Ipilimumab, Nivolumab, Pembrolizumab, Avelumab, Durvalumab, and Atezolizumab. Additional exemplary immune checkpoint inhibitors include cemiplimab, tremelimumab, durvalumab, spartalizumab, relatlimab, IMP321, LAG525, MBG453, MEDI9447, and enoblitzumab.

Yet other agents that may be used as part of a combination therapy in treating cancer are agents that target cancer-associated fibroblasts or cancer-associated extracellular matrix. These agents include, for example: a fibroblast activation protein alpha (FAP) blocking antibody such as sibrotuzumab, or a FAP vaccine, pegvorhyaluronidase alfa (PEGPH20), a CD44 targeting antibody such as RG7356/RO5429083, metformin, a lysyl oxidase inhibitor or blocking antibody such as simtuzumab, a TGF-beta blocking antibody such as fresolimumab, and a TGF-beta receptor inhibitor such as galunisertib.

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

Yet other categories of anti-cancer agents include, for example: (i) an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDCl₇ Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

Antibodies of the invention can also be used as an adjunct to surgical removal of cancer from the primary lesion.

Exemplary therapeutic agents that may be used as a part of a combination therapy with the anti-TSG-6 antibodies for treating fibrosis include, for example, nintedanib, pirfenidone, corticosteroids, proton pump inhibitors, metformin, a lysyl oxidase inhibitor or blocking antibody such as simtuzumab, a TGF-beta blocking antibody such as fresolimumab, and a TGF-beta receptor inhibitor such as galunisertib.

Exemplary therapeutic agents that may be used as a part of a combination therapy with the anti-TSG-6 antibodies for treating, delaying the progression of, preventing a relapse of, or alleviating a symptom of an autoimmune or inflammatory disorder, include, for example, any of a variety of known anti-inflammatory and/or immunosuppressive therapies. In some embodiments, the anti-inflammatory and/or immunosuppressive therapies include, but are not limited to methotrexate, cyclosporin A (including, for example, cyclosporin microemulsion), tacrolimus, corticosteroids, statins, interferon beta, non-steroidal anti-inflammatory agents, and 6-MP (Mercaptopurine, also called 6-Mercaptopurine, or Purinethol).

In some embodiments, the anti-inflammatory and/or immunosuppressive therapies for combining with the anti-TSG-6 antibodies include, but are not limited to a TOPK inhibitor (e.g., OTS964 ((R)-9-(4-(1-(dimethylamino)propan-2-yl)phenyl)-8-hydroxy-6-methylthieno[2, 3-c] quinolin-4(5H)-one) (Oncotherapy Science)), a tyrosine kinase inhibitor (e.g., axitinib, dasatinib, icotinib), a topoisomerase inhibitor (e.g., topotecan), a sphingosine-1-phosphate receptor agonist (e.g., fingolimod, KRP-203), anti-T cell immunoglobulin (e.g. AtGam), anti-IL-2 receptor antibody (e.g. daclizumab), amides (CTX), ifosfamide (IFO), adriamycin (ADM), daunorubicin (DNR), vincristine (VCR), vinblastine (VBL), etoposide (VP16), vermeer (Vumon), carboplatin (CBP), tacrolimus, sirolimus, everolimus, azathioprine, brequinar, leflunomide, LEA-29Y, anti-CD3 antibody (e.g. OKT3), aspirin, B7-CD28 blocking molecules (e.g. belatacept, abatacept), CD40-CD154 blocking molecules (anti-CD40 antibodies), acetaminophen, ibuprofen, naproxen, piroxicam, and anti-inflammatory steroids (e.g. prednisolone or dexamethasone).

In some embodiments, the anti-inflammatory and/or immunosuppressive therapies for combining with the anti-TSG-6 antibodies include ablation of autoimmune cells, for example, by administration of TNF-alpha, CFA, interleukin-1 (IL-1), proteasome inhibitors, NFκB inhibitors, anti-inflammatory drugs, tissue plasminogen activator (TPA), lipopolysaccharide, UV light, and an intracellular mediator of the TNF-alpha signaling pathway. Such agents induce the apoptosis of autoreactive lymphocytes by interrupting the pathway downstream from TNF-alpha receptor signaling or act downstream of TNF-alpha receptor binding. (Baldwin et al., Ann. Rev. Immunol. (1996) 12:141; Baltimore, Cell (1996) 87:13).

Exemplary therapeutic agents that may be used as a part of a combination therapy with the anti-TSG-6 antibodies for treating idiopathic pulmonary artery hypertension include, for example, calcium channel blockers, vasodilators, prostacyclin pathway agonists, endothelin receptor antagonists, nitric oxide (NO)-cGMP enhancers, blood thinners, diuretics, digoxin, anticoagulation therapy, and surgery.

Exemplary therapeutic agents that may be used as a part of a combination therapy with the anti-TSG-6 antibodies for treating idiopathic pulmonary artery hypertension may also include treating the underlying fibrosis with, for example, nintedanib, pirfenidone, corticosteroids, proton pump inhibitors, metformin, a lysyl oxidase inhibitor or blocking antibody such as simtuzumab, a TGF-beta blocking antibody such as fresolimumab, and a TGF-beta receptor inhibitor such as galunisertib.

In other embodiments, the anti-inflammatory and/or immunosuppressive therapies for combining with anti-TSG-6 antibodies include but are not limited to short-acting β2-agonists, long-acting β2-agonists, anticholinergics, corticosteroids, systemic corticosteroids, mast cell stabilizers, leukotriene modifiers, methylxanthines, β2-agonists, albuterol, levalbuterol, pirbuterol, artformoterol, formoterol, salmeterol, anticholinergics including ipratropium and tiotropium; corticosteroids including beclomethasone, budesonide, flunisolide, fluticasone, mometasone, triamcinolone, methyprednisolone, prednisolone, prednisone; leukotriene modifiers including montelukast, zafirlukast, and zileuton; mast cell stabilizers including cromolyn and nedocromil; methylxanthines including theophylline; combination drugs including ipratropium and albuterol, fluticasone and salmeterol, budesonide and formoterol; antihistamines including hydroxyzine, diphenhydramine, loratadine, cetirizine, and hydrocortisone; immune system modulating drugs including tacrolimus and pimecrolimus; cyclosporine; azathioprine; mycophenolatemofetil; and combinations thereof.

The amount of the antibodies and additional therapeutic agents and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

VI. Pharmaceutical Composition and Administration

The present disclosure also features pharmaceutical compositions/formulations that contain a therapeutically effective amount of an anti-TSG-6 antibody described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The antibodies of the present disclosure can exist in a lyophilized formulation or liquid aqueous pharmaceutical formulation. The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The antibodies of the present disclosure could exist in a lyophilized formulation including the proteins and a lyoprotectant. The lyoprotectant may be sugar, e.g., disaccharides. In certain embodiments, the lyoprotectant is sucrose or maltose. The lyophilized formulation may also include one or more of a buffering agent, a surfactant, a bulking agent, and/or a preservative.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. It may be administered in the range of 0.1 mg to 1 g and preferably in the range of 0.5 mg to 500 mg of active antibody per administration for adults. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### EXAMPLES

The invention now being generally described, will be more readily understood by reference to the following examples, which are included merely for purposes of illustration of certain aspects and embodiments of the present invention, and is not intended to limit the invention.

### Example 1—Generation of TSG-6 Antibodies

Mouse antibodies directed against mouse TSG-6 protein (mTSG-6) were generated using conventional mouse monoclonal antibody techniques. Briefly, mice (strain SJL/J) were inoculated with KLH-coupled recombinant mouse TSG-6 (R&D Biosystems, catalog #2326-TS). After boosting, antibody titers were determined by ELISA, using a non-relevant His6-tagged protein as a control. Mice selected for fusion were sacrificed and hybridoma fusion was performed using the P3X63Ag8.653 murine myeloma cell line as a fusion partner. Clones were isolated by limiting dilution and selected by ELISA of hybridoma supernatants. Recombinant human TSG-6 (R&D Biosystems, catalog #2104-TS) was used as a substrate for ELISA assays thus facilitating the selection of human- and mouse-cross reactive clones (the two proteins are 92% identical at the amino acid level). Hybridoma mRNA was transcribed to cDNA and amplified by 5′-rapid amplification of cDNA ends (RACE). PCR products were cloned into an appropriate sequencing vector and sequenced by the dideoxy Sanger method. Multiple copies were sequenced to verify the clonality of each clone. Translated protein sequences were entered into the NCBI IgBLAST search tool (Ye, J., et al., 2013, Nucleic Acids Res. 41, W34-40) to identify CDRs.

### Example 2—Inhibition of HA Binding to TSG-6 by Anti-TSG-6 Antibodies

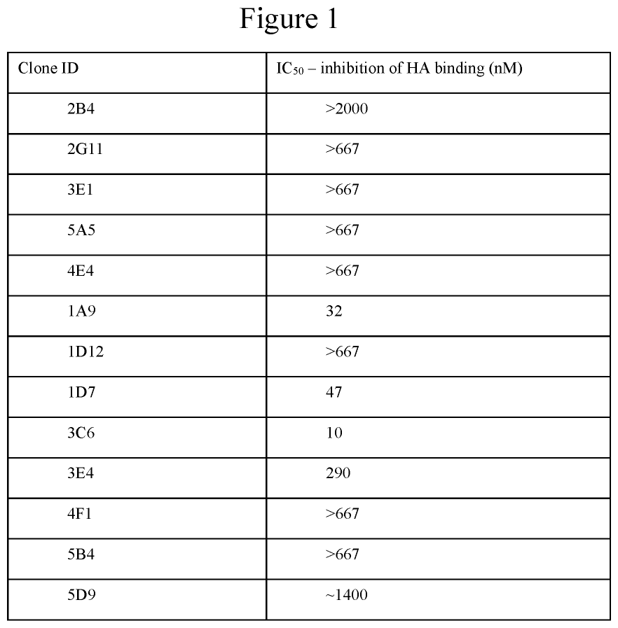

Recombinant human TSG-6 was immobilized on 96-well polystyrene microtiter plates (MaxiSorp, Nunc) at 100 ng/well. Wells were blocked with 1% BSA in PBS. Wells were incubated with hyaluronan-biotin (Sigma, B1557) at 1 μg/mL in the presence of various concentrations of anti-TSG-6 antibodies. After washing plates with TBST, biotin binding to TSG-6 was detected with avidin-HRP (e-Biosciences, 18-4100-51), and color was developed with TMB ELISA substrate solution (e-Biosciences, 00-4201-56). Color development was stopped with 1 M H3PO4 and plates were read on a SpectraMax M5 plate reader (Molecular Devices). (FIG. 1) IC50 values were determined using a 4-parameter logistic model (XLfit, IDBS).

### Example 3—Inhibition of TSG-6 HC-HA Transesterase Activity by Anti-TSG-6 Antibodies

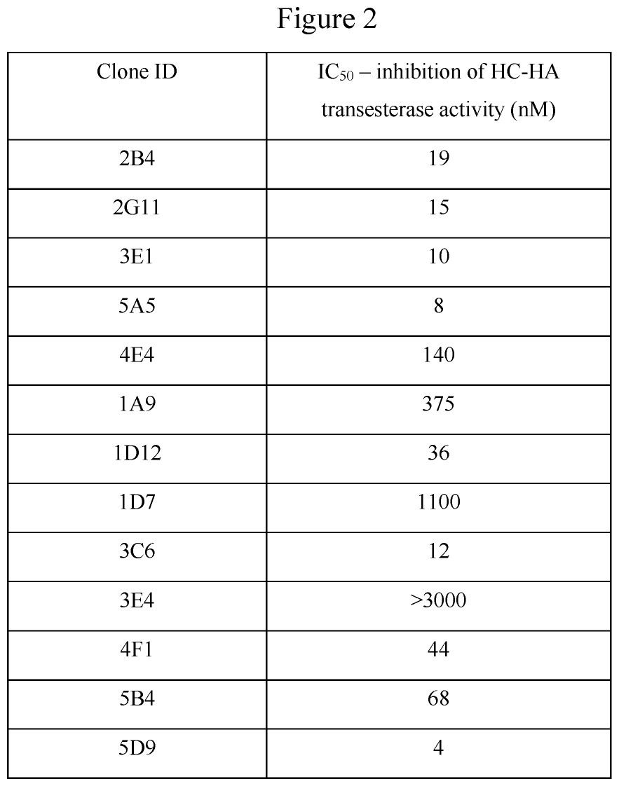

Recombinant human TSG-6 (1 nM in PBS) was incubated in the presence of 1% human plasma (as a source of IαI), 5 mM MgCl2, 1 μg/mL HA and various concentrations of anti-TSG-6 antibodies at 37° C. for 1-2 h. EDTA (10 mM) was used as a control for 100% inhibition of the transesterase activity, which is metal ion dependent. These incubations were then loaded onto 96-well polystyrene microtiter plates (MaxiSorp, Nunc) pre-coated with bovine cartilage hyaluronan binding protein (Millipore, 385910) at 100 ng/well and blocked with 1% BSA in PBS. After incubation for 1 h at RT, plates were rinsed with PBST, then HC-HA was detected with anti-HC (ITIH1) antibody (Abnova, H00003697) followed by HRP-linked anti-rabbit antibody (GE Life Sciences, NA934). Color was developed with TMB ELISA substrate solution (e-Biosciences, 00-4201-56), and stopped with 1 M H3PO4. Plates were read on a SpectraMax M5 plate reader (Molecular Devices) and IC50 values were determined using a 4-parameter logistic model (XLfit, IDBS). (FIG. 2)

### Example 4—Demonstration of an Anti-TSG-6 Antibody Pharmacodynamic Assay

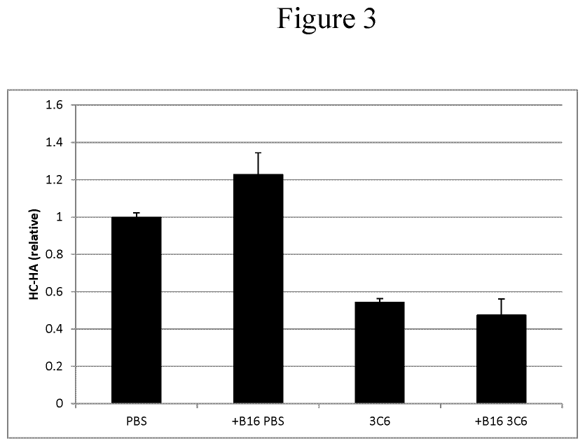

C57Bl/6 mice (n=3 per group) were left un-inoculated or inoculated with B16F0 melanoma cells (1×10⁶) 1 day prior to treatment. Treatment was with PBS (vehicle) or antiTSG-6 antibody 3C6 intraperitoneally at a fixed dose of 1 mg. Ten days after dosing, serum samples were collected and diluted 1:10 with PBS. These samples were then loaded onto 96-well polystyrene microtiter plates (MaxiSorp, Nunc) pre-coated with bovine cartilage hyaluronan binding protein (Millipore, 385910) at 100 ng/well and blocked with 1% BSA in PBS. After incubation for 1 h at RT, plates were rinsed with PBST, then HC-HA was detected with anti-HC (ITIH1) antibody (Abnova, H00003697) followed by HRP-linked anti-rabbit antibody (GE Life Sciences, NA934). Color was developed with TMB ELISA substrate solution (e-Biosciences, 00-4201-56), and stopped with 1 M H3PO4. (FIG. 3) (One repeat was removed from each of the non-inoculated untreated group and the non-inoculated treated group because of a technical error; these outliers came from adjacent wells on the plate layout).

### Example 5—Anti-Tumor Activity of the Anti-TSG-6 Antibody 3C6 in Tumors Arising from B16F0 Melanoma Cells Co-Injected with CAFs

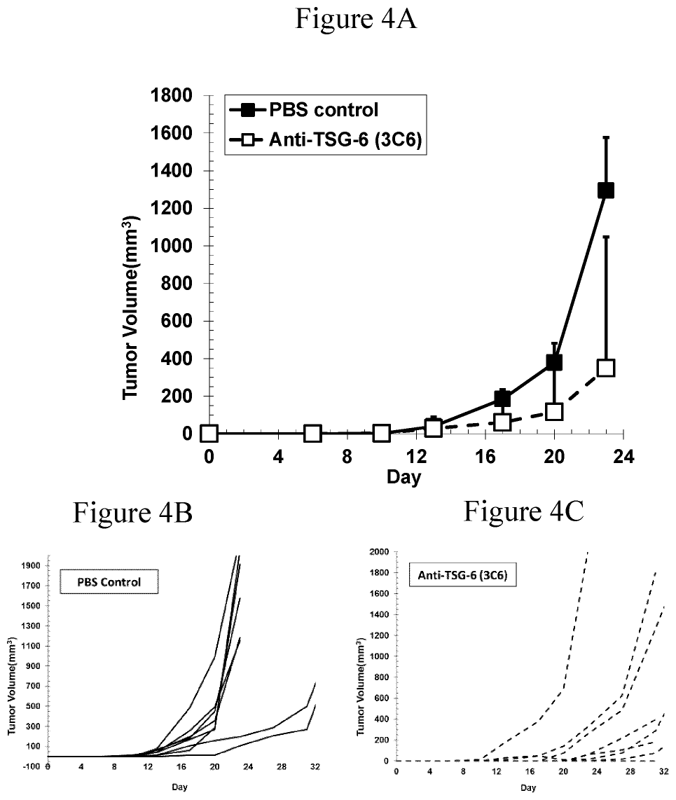

CAFs were isolated from conventionally-grown B16F0 tumors (106 cells inoculated, excised at 1500 mm3) by disaggregating tumors followed by sorting for cells negative for epithelial markers, endothelial markers and leukocyte markers, and positive for PDGFRa using magnetic sorting (Miltenyi Biotec). These CAFs were then co injected with B16F0 melanoma cells (1000 B16F0: 1000 CAF cells) into naïve female C57Bl/6 mice. Treatment was initiated at the time of cell inoculation (day 0). Treatment was either with PBS (vehicle control) or anti-TSG-6 antibody 3C6 (1 mg fixed dose) every 7 days (n=8 per group) (FIG. 4A-4C). Individual tumor growth curves are shown at right.

### Example 6—Anti-Tumor Activity of the Anti-TSG-6 Antibody 3C6, and its Combination Activity with Anti-PD-1

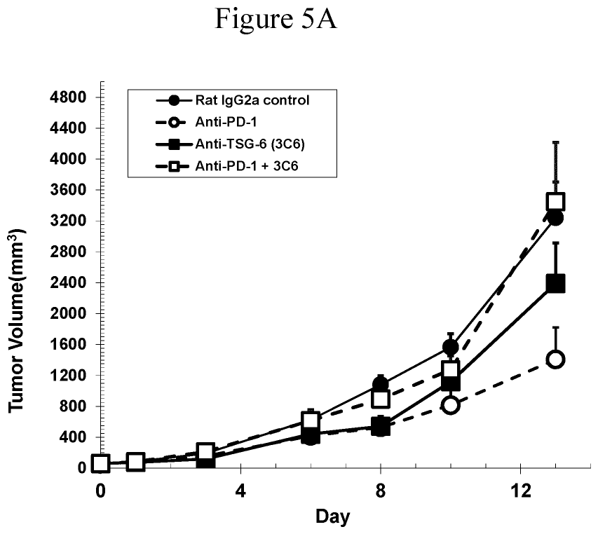

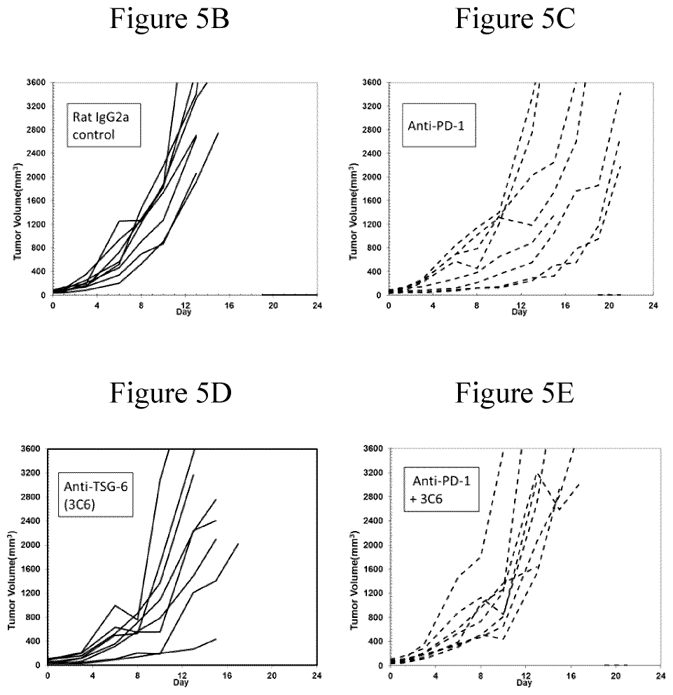

Mice (C57Bl/6, female, n=8 per group) were inoculated with B16F0 melanoma cells six days prior to treatment. Mice were treated with either rat IgG2a as a negative control (same isotype as the anti-PD-1 antibody), anti-Mouse PD-1 (CD279) RMP1-14 (BioXcell) 150 μg days 0, 3, 6, 10), anti-TSG-6 antibody 3C6 (1 mg days 0, 7) or a combination. Individual tumor growth curves are shown at right. IL-2 in the supernatant was measured by ELISA using anti-IL2 capture antibody (R&D system MAB602) (FIG. 5A-5E).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. An antibody comprising:
a) a heavy chain variable region comprising an amino acid sequence of SEQ ID NO:1 and a light chain variable region comprising an amino acid sequence of SEQ ID NO:2;
b) a heavy chain variable region comprising an amino acid sequence of SEQ ID NO:3 and a light chain variable region comprising an amino acid sequence of SEQ ID NO:4;
c) a heavy chain variable region comprising an amino acid sequence of SEQ ID NO:5 and a light chain variable region comprising an amino acid sequence of SEQ ID NO:6;
d) a heavy chain variable region comprising an amino acid sequence of SEQ ID NO:7 and a light chain variable region comprising an amino acid sequence of SEQ ID NO:8;
e) a heavy chain variable region comprising an amino acid sequence of SEQ ID NO:9 a light chain variable region comprising an amino acid sequence of SEQ ID NO:10;
f) a heavy chain variable region comprising an amino acid sequence of SEQ ID NO:11 and a light chain variable region comprising an amino acid sequence of SEQ ID NO:12;
g) a heavy chain variable region comprising an amino acid sequence of SEQ ID NO:13 and a light chain variable region comprising an amino acid sequence of SEQ ID NO:14;
8) a heavy chain variable region comprising an amino acid sequence of SEQ ID NO:15 and a light chain variable region comprising an amino acid sequence of SEQ ID NO:16;
h) a heavy chain variable region comprising an amino acid sequence of SEQ ID NO:17 and a light chain variable region comprising an amino acid sequence of SEQ ID NO:18;
i) a heavy chain variable region comprising an amino acid sequence of SEQ ID NO:19 and a light chain variable region comprising an amino acid sequence of SEQ ID NO:20;
j) a heavy chain variable region comprising an amino acid sequence of SEQ ID NO:21 and a light chain variable region comprising an amino acid sequence of SEQ ID NO:22;
k) a heavy chain variable region comprising an amino acid sequence of SEQ ID NO:23 and a light chain variable region comprising an amino acid sequence of SEQ ID NO:24;
l) a heavy chain variable region comprising an amino acid sequence of SEQ ID NO:25 and a light chain variable region comprising an amino acid sequence of SEQ ID NO:26;

2. An antibody comprising:
a) a vhCDR1 comprising SEQ ID NO:27, a vhCDR2 comprising SEQ ID NO:28, a vhCDR3 comprising SEQ ID NO:29, a vlCDR1 comprising SEQ ID NO:30, a vlCDR2 comprising SEQ ID NO:31, and a vlCDR3 comprising SEQ ID NO:32;
b) a vhCDR1 comprising SEQ ID NO:33, a vhCDR2 comprising SEQ ID NO:34, a vhCDR3 comprising SEQ ID NO:35, a vlCDR1 comprising SEQ ID NO:36, a vlCDR2 comprising SEQ ID NO:37, and a vlCDR3 comprising SEQ ID NO:38;
c) a vhCDR1 comprising SEQ ID NO:39, a vhCDR2 comprising SEQ ID NO:40, a vhCDR3 comprising SEQ ID NO:41, a vlCDR1 comprising SEQ ID NO:42, a vlCDR2 comprising SEQ ID NO:43, and a vlCDR3 comprising SEQ ID NO:44;
d) a vhCDR1 comprising SEQ ID NO:45, a vhCDR2 comprising SEQ ID NO:46, a vhCDR3 comprising SEQ ID NO:47, a vlCDR1 comprising SEQ ID NO:48, a vlCDR2 comprising SEQ ID NO:49, and a vlCDR3 comprising SEQ ID NO:50;
e) a vhCDR1 comprising SEQ ID NO:51, a vhCDR2 comprising SEQ ID NO:52, a vhCDR3 comprising SEQ ID NO:53, a vlCDR1 comprising SEQ ID NO:54, a vlCDR2 comprising SEQ ID NO:55, and a vlCDR3 comprising SEQ ID NO:56;
f) a vhCDR1 comprising SEQ ID NO:57, a vhCDR2 comprising SEQ ID NO:58, a vhCDR3 comprising SEQ ID NO:59, a vlCDR1 comprising SEQ ID NO:60, a vlCDR2 comprising SEQ ID NO:61, and a vlCDR3 comprising SEQ ID NO:62;
g) a vhCDR1 comprising SEQ ID NO:63, a vhCDR2 comprising SEQ ID NO:64, a vhCDR3 comprising SEQ ID NO:65, a vlCDR1 comprising SEQ ID NO:66, a vlCDR2 comprising SEQ ID NO:67, and a vlCDR3 comprising SEQ ID NO:68;
h) a vhCDR1 comprising SEQ ID NO:69, a vhCDR2 comprising SEQ ID NO:70, a vhCDR3 comprising SEQ ID NO:71, a vlCDR1 comprising SEQ ID NO:72, a vlCDR2 comprising SEQ ID NO:73, and a vlCDR3 comprising SEQ ID NO:74;
i) a vhCDR1 comprising SEQ ID NO:75, a vhCDR2 comprising SEQ ID NO:76, a vhCDR3 comprising SEQ ID NO:77, a vlCDR1 comprising SEQ ID NO:78, a vlCDR2 comprising SEQ ID NO:79, and a vlCDR3 comprising SEQ ID NO:80;
j) a vhCDR1 comprising SEQ ID NO:81, a vhCDR2 comprising SEQ ID NO:82, a vhCDR3 comprising SEQ ID NO:83, a vlCDR1 comprising SEQ ID NO:84, a vlCDR2 comprising SEQ ID NO:85, and a vlCDR3 comprising SEQ ID NO:86;
k) a vhCDR1 comprising SEQ ID NO:87, a vhCDR2 comprising SEQ ID NO:88, a vhCDR3 comprising SEQ ID NO:89, a vlCDR1 comprising SEQ ID NO:90, a vlCDR2 comprising SEQ ID NO:91, and a vlCDR3 comprising SEQ ID NO:92;
l) a vhCDR1 comprising SEQ ID NO:93, a vhCDR2 comprising SEQ ID NO:94, a vhCDR3 comprising SEQ ID NO:95, a vlCDR1 comprising SEQ ID NO:96, a vlCDR2 comprising SEQ ID NO:97, and a vlCDR3 comprising SEQ ID NO:98;
m) a vhCDR1 comprising SEQ ID NO:99, a vhCDR2 comprising SEQ ID NO:100, a vhCDR3 comprising SEQ ID NO:101, a vlCDR1 comprising SEQ ID NO:102, a vlCDR2 comprising SEQ ID NO:103, and a vlCDR3 comprising SEQ ID NO:104;

3. The antibody according to any of the previous claims, wherein the antibody binds human and/or mouse TSG-6.

4. The antibody according to any one of the previous claims, wherein the antibody comprises a constant region with an amino acid sequence at least 90% identical to a human IgG.

5. The antibody according to claim 4, wherein the human IgG is selected from a group consisting of IgG1, IgG2, IgG3 and IgG4.

6. The antibody according to claim 5, wherein the IgG is an IgG1.

7. The antibody according to claim 5, wherein the IgG is an IgG2b.

8. A nucleic acid composition encoding the antibody according to any one of the previous claims.

9. An expression vector composition comprising the nucleic acid composition according to claim 8, wherein the first nucleic acid is contained in a first expression vector and the second nucleic acid is contained in a second expression vector.

10. An expression vector composition comprising the nucleic acid composition according to claim 8, wherein the first nucleic acid and the second nucleic acid are contained in a single expression vector.

11. A host cell comprising the expression vector composition of claim 9 or 10.

12. A method of making an antibody comprising culturing said host cell of claim 11 under conditions wherein the antibody is expressed, and recovering the antibody.

13. A composition comprising the antibody according to any one of claims 1-8, and a pharmaceutical acceptable carrier or diluent.

14. A method of modulating an immune response in a subject, the method comprising administering to the subject an effective amount of the antibody according to any one of the claims 1-8 or the composition according to claim 13.

15. The method of claim 14, wherein the method stimulates an immune response in a subject, the method comprising administering to the subject an effective amount of the antibody according to any one of the claims 1-8 or the composition according to claim 13, wherein the antibody serves as a TSG-6 antagonist.

16. The method of claim 14, wherein the method suppresses an immune response in a subject, the method comprising administering to the subject an effective amount of the antibody according to any one of the claims 1-8 or the composition according to claim 13, wherein the antibody serves as a TSG-6 agonist.

17. A method of treating cancer in a subject comprising administering to the subject an effective amount of the antibody according to any one of the claims 1-8 or the composition according to claim 13, wherein the antibody serves as a TSG-6 antagonist.

18. The method of claim 17, wherein the cancer has high expression of TSG-6 and/or HC-HA.

19. The method of claim 15, 17, or 18, wherein the subject to be treated has high expression of TSG-6 and/or HC-HA.

20. The method according to any one of claims 17-19, wherein the cancer is a solid tumor.

21. The method according to any one of claims 17-20, wherein the cancer is melanoma.

22. The method according to any one of the claims 17-21, wherein the antibody is combined with one or more additional therapeutic agents to treat cancer.

23. The method of claim 22, wherein the additional therapeutic agents are other immune checkpoint inhibitors.

24. The method of claim 23, wherein the other immune checkpoint inhibitors are selected from the group consisting of a PD-1 inhibitor, a PD-L1 inhibitor, a CTLA-4 inhibitor, a TIM-3 inhibitor, and a LAG-3 inhibitor.

25. A method of treating fibrosis in a subject comprising administering to the subject an effective amount of the antibody according to any one of the claims 1-8 or the composition according to claim 13, wherein the antibody serves as a TSG-6 antagonist.

26. The method of claim 25, wherein the fibrotic tissue has high expression of TSG-6 and/or HC-HA.

27. The method of claim 25 or 26, wherein the subject to be treated has high expression of TSG-6 and/or HC-HA.

28. The method of any one of claims 25-27, wherein the antibody is combined with one or more additional therapeutic agents to treat fibrosis.

29. A method of treating an autoimmune or inflammatory disorder in a subject comprising administering to the subject an effective amount of the antibody according to any one of the claims 1-8 or the composition according to claim 13, wherein the antibody serves as a TSG-6 antagonist.

30. The method of claim 29, wherein the autoimmune or inflammatory disorder is idiopathic pulmonary artery hypertension.

31. The method of claim 29 or 30, wherein the subject to be treated has high expression of TSG-6 and/or HC-HA.

32. The method of claim 30 or 31, wherein the subject to be treated also has lung fibrosis.

33. The method of any one of claims 29-32, wherein the antibody is combined with one or more additional therapeutic agents to treat pulmonary artery hypertension and/or lung fibrosis.

34. The method of claim 29, wherein the autoimmune or inflammatory disorder is asthma.

35. The method of claim 34, wherein the subject to be treated has high expression of TSG-6 and/or HC-HA.

36. The method of claim 34 or 35, wherein the subject to be treated has high expression of TSG-6 and/or HC-HA in the lung.

37. The method of any one of claims 34-36, wherein the subject to be treated has increased airway eosinophilia.

38. A method of treating an autoimmune or inflammatory disorder in a subject comprising administering to the subject an effective amount of the antibody according to any one of the claims 1-8 or the composition according to claim 13, wherein the antibody serves as a TSG-6 agonist.

39. The method of claim 38, wherein the autoimmune or inflammatory disorder is rheumatoid arthritis.

40. The method of any one of claim 16, 38, or 39, wherein the subject to be treated has low expression of TSG-6 and/or HC-HA.

41. The method of any one of claims 29-40, wherein the antibody is combined with one or more additional therapeutic agents to treat an autoimmune or inflammatory disorder.

42. An antibody comprising:
a) a heavy chain variable region comprising an amino acid sequence of SEQ ID NO:17 and a light chain variable region comprising an amino acid sequence of SEQ ID NO:18;

43. An antibody comprising:
a) a vhCDR1 comprising SEQ ID NO:75, a vhCDR2 comprising SEQ ID NO:76, a vhCDR3 comprising SEQ ID NO:77, a vlCDR1 comprising SEQ ID NO:78, a vlCDR2 comprising SEQ ID NO:79, and a vlCDR3 comprising SEQ ID NO:80;

44. The antibody according to claim 42 or 43, wherein the antibody binds human and/or mouse TSG-6.

45. The antibody according to any one claims 42-44, wherein the antibody comprises a constant region with an amino acid sequence at least 90% identical to a human IgG.

46. The antibody according to claim 45, wherein the human IgG is selected from a group consisting of IgG1, IgG2, IgG3 and IgG4.

47. The antibody according to claim 46, wherein the IgG is an IgG1.

48. A nucleic acid composition encoding the antibody according to any one of claims 42-47.

49. An expression vector composition comprising the nucleic acid composition according to claim 48, wherein the first nucleic acid is contained in a first expression vector and the second nucleic acid is contained in a second expression vector.

50. An expression vector composition comprising the nucleic acid composition according to claim 48, wherein the first nucleic acid and the second nucleic acid are contained in a single expression vector.

51. A host cell comprising the expression vector composition of claim 49 or 50.

52. A method of making an antibody comprising culturing said host cell of claim 51 under conditions wherein the antibody is expressed, and recovering the antibody.

53. A composition comprising the antibody according to any one of claims 42-48, and a pharmaceutical acceptable carrier or diluent.

54. A method of modulating an immune response in a subject, the method comprising administering to the subject an effective amount of the antibody according to any one of the claims 42-48 or the composition according to claim 53.

55. The method of claim 54, wherein the method stimulates an immune response in a subject, the method comprising administering to the subject an effective amount of the antibody according to any one of the claims 42-48 or the composition according to claim 53, wherein the antibody serves as a TSG-6 antagonist.

56. A method of treating cancer in a subject comprising administering to the subject an effective amount of the antibody according to any one of the claims 42-48 or the composition according to claim 53, wherein the antibody serves as a TSG-6 antagonist.

57. The method of claim 56, wherein the cancer has high expression of TSG-6 and/or HC-HA.

58. The method of any one of claims 55-57, wherein the subject to be treated has high expression of TSG-6 and/or HC-HA.

59. The method according to any one of claims 56-58, wherein the cancer is a solid tumor.

60. The method according to any one of claims 56-59, wherein the cancer is melanoma.

61. The method according to any one of claims 56-60, wherein the antibody is combined with one or more additional therapeutic agents to treat cancer.

62. The method of claim 61, wherein the additional therapeutic agents are other immune checkpoint inhibitors.

63. The method of claim 62, wherein the other immune checkpoint inhibitors are selected from the group consisting of a PD-1 inhibitor, a PD-L1 inhibitor, a CTLA-4 inhibitor, a TIM-3 inhibitor, and a LAG-3 inhibitor.

64. A method of treating fibrosis in a subject comprising administering to the subject an effective amount of the antibody according to any one of the claims 42-48 or the composition according to claim 53, wherein the antibody serves as a TSG-6 antagonist.

65. The method of claim 64, wherein the fibrotic tissue has high expression of TSG-6 and/or HC-HA.

66. The method of claim 64 or 65, wherein the subject to be treated has high expression of TSG-6 and/or HC-HA.

67. The method of any one of claims 64-66, wherein the antibody is combined with one or more additional therapeutic agents to treat fibrosis.

68. A method of treating an autoimmune or inflammatory disorder in a subject comprising administering to the subject an effective amount of the antibody according to any one of the claims 42-48 or the composition according to claim 53, wherein the antibody serves as a TSG-6 antagonist.

69. The method of claim 68, wherein the autoimmune or inflammatory disorder is idiopathic pulmonary artery hypertension.

70. The method of claim 68 or 69, wherein the subject to be treated has high expression of TSG-6 and/or HC-HA.

71. The method of claim 69 or 70, wherein the subject to be treated also has lung fibrosis.

72. The method of any one of claims 69-71, wherein the antibody is combined with one or more additional therapeutic agents to treat pulmonary artery hypertension and/or lung fibrosis.

73. The method of claim 68, wherein the autoimmune or inflammatory disorder is asthma.

74. The method of claim 73, wherein the subject to be treated has high expression of TSG-6 and/or HC-HA.

75. The method of claim 73 or 74, wherein the subject to be treated has high expression of TSG-6 and/or HC-HA in the lung.

76. The method of any one of claims 73-75, wherein the subject to be treated has increased airway eosinophilia.

77. The method of any one of claims 68-76, wherein the antibody is combined with one or more additional therapeutic agents to treat an autoimmune or inflammatory disorder.


SEQUENCE LISTING (104 sequences)

SEQ ID NO: 1 - protein, 262 aa, Artificial Sequence
  Synthetic polypeptide
MERHWIFLSLLSVIAGVHSQVRLQQSGAELAKPGASVKLSCKASGYTFTSYWMHWVKQRPGQGLEWIGYIIPSSGYTKNKQNFKDKATLTADKSSSTAYMQLSNLTYEDSAVYSCARSDGSYPYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPK

SEQ ID NO: 2 - protein, 225 aa, Artificial Sequence
  Synthetic polypeptide
MDFQVQIFSFLLISASVIMSRGENVLTQSPAIMSASPGEKVTMTCSASSSVSYMHWYQQKSSTSPKLWIYDTSKLASGVPGRFSGSGSGNSYSLTISSMEAEDVATYYCFQGSGYPLTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 3 - protein, 287 aa, Artificial Sequence
  Synthetic polypeptide
MGWSWIFLFLLSGTAGVHSQVQLQQSGPELVKPGASVKLSCKASGYTFTSYDINWVKQRPGQGLEWIGWIYPRDGSTKYNEKFKDKATWTIDTSSNRAYMEIHSLTSEDSAVYFCARGLWYYVSGMDYWGPGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 4 - protein, 228 aa, Artificial Sequence
  Synthetic polypeptide
MKLPVRLLVLMFWIPASSSDVVMTQTPLSLPVSLGDQASISCRSSQSLVHSNGNTYLHWYLQKPGQSPKLLIYKVSNRFSGVPDRFSGRGSGIDFTLKISRVEAEDLGVYFCSQSTHVPPTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 5 - protein, 287 aa, Artificial Sequence
  Synthetic polypeptide
MERHWIFLSLLSVIAGVHSQVQLQQSGAELAKPGASVRLSCQASGYTFTSYWMHWVKQRPRQGLEWIGYIIPSSGYTKCHQKFKDKATLTADKSSSTAYMQLSSLTYEDSAVYYCARSTSGYPYYFDSWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 6 - protein, 225 aa, Artificial Sequence
  Synthetic polypeptide
MDFQVQIFSFLLISASVIMSRGENVLTQSPAIMSASPGERVTMTCSATSSVSFMHWYQQKSSTSPKLWIYDTSKLASGVPGRFSGSGSGNSYSLTISSMEAEDVATYYCFQGSWYPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 7 - protein, 287 aa, Artificial Sequence
  Synthetic polypeptide
MERHWIFLSLLSVIAGVHSQVQLQQSGAELAKPGASVRLSCQASGYTFTTYWMHWVKQRPRQGLEWIGYIIPSSGYSKCHQKFKDKATLTADKSSNTASMQLSSLTYEDSAVYYCARSTSGYPYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 8 - protein, 225 aa, Artificial Sequence
  Synthetic polypeptide
MDFQVQIFSFLLISASVIMSRGENVLTQSPAIMSASPGEKVTMTCSATSSVSYMHWYQQKSSTSPKLWIYDTSKLASGVPGRFSGSGSGNSYSLTISSMEAEDVATYYCFQGSWYPLTFGAGTKLELKRADAAPTVSIFPPSSEQLTSGGASVVCFLNNFYPRDINVKWKIDGSERQDGVLNSWTDQDSKDSTYSMSSTLTLTKDEYERHNSYTCEATHKTSTSP

SEQ ID NO: 9 - protein, 285 aa, Artificial Sequence
  Synthetic polypeptide
MGWSYIILFLVATATGVHSQVQLQQPGAELVKPGASVKLSCKASGYTFTSYWMHWVKQRPGQGLEWIGMIHPNSGSTNYNEKFKNKATLTVDKSSSTAYMQLSSLTSEDSAVYYCAPRMFDDYDDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 10 - protein, 226 aa, Artificial Sequence
  Synthetic polypeptide
DFQVQIFSFLLISASVIMSRGQIVLTQSPAIMSASLGERVTMTCTASSSVSSSYLHWYQQKPGSSPKLWIYSTSNLASGVPVRFSGSGSGTSYSLTISRMEAEDAATYYCHHYHRSPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 11 - protein, 286 aa, Artificial Sequence
  Synthetic polypeptide
MGWSWIFLFLLSGTAGVLSEVQLQQSGPELVKPGASVKISCKASGYTFTDYYMNWVKQSHEKSLEWIGDINPNNGGTSYNQKFMGKATLTVDKSSRTAYMELRSLTSEDSTVYYCARWGIYDRFTYWGQGTLVSVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQG

SEQ ID NO: 12 - protein, 226 aa, Artificial Sequence
  Synthetic polypeptide
MDMRTPAQFLGILLLWFPGIKCDIKMTQSPSSMYASLGERVTIACKASQDINSFLSWFQQKPGKSPKTLIYRANRLVDGVPSRFSGSGSGQDYSLTISSLEYEDMGIYYCLQYDEFPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 13 - protein, 287 aa, Artificial Sequence
  Synthetic polypeptide
MGWSWIFLFLLSGTAGVHSQVQLQQSGPELVKPGASVKLSCKTSGYTFTSYDINWVKQRPGQGLEWIGWIYPSDGSTKFNEKFKGMATLTVDTSSSTAYMELHSLTSEDSTVYFCARGLWYYGGGVDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 14 - protein, 228 aa, Artificial Sequence
  Synthetic polypeptide
MKLPVRLLVLMFWIPASSSDAVMTQTPLSLPVSLGDQASISCRSTQSLEDSNGNTYLNWYLQKPGQSPQLLIYRVSNRFSGVLDRFSGSGSGTDFTLKISRVEAEDLGVYFCLQVTHVPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 15 - protein, 285 aa, Artificial Sequence
  Synthetic polypeptide
MGWSWIFLFLLSGTAGVLSEVQLQQSGPELVKPGASVKISCKASGYTFTDYYMNWVKQSHGKSLEWIGDINPNNGGTTYNQKFKGKATLTVDKSSSTAYMELRSLTSEDSAVYYCARWGIFDRFTYWGQGTVVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 16 - protein, 225 aa, Artificial Sequence
  Synthetic polypeptide
MDMRTPAQFLGILLLWFPGIKCDIKMTQSPSSMYASRGERVTITCKASQDINSFLSWFQQKPGKSPKTLIYRANRLVDGVPSRFSGSGSGQDYSLTISSLEYEDMGIYYCLQYDEFPLTFGAGTKLELKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTK

SEQ ID NO: 17 - protein, 259 aa, Artificial Sequence
  Synthetic polypeptide
MGWSWIFLLSLPGTAGVLSEVQLQQSGPELVKPGASVKIPCKASGYTFTDYNMDWVKQSHGESLEWIGDIYPNNGGTIYNQKFKDKATLTVDKSSSTAYMELRSLTSEDNAVYYCARKTGTGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPK

SEQ ID NO: 18 - protein, 225 aa, Artificial Sequence
  Synthetic polypeptide
MDFQVQIFSFLLISASVIMSRGQIVLSQSPAILSASPGEKVTMTCRARSSVIYMHWYQQKPGSSPKPWIYATFNLASGVPARFSGSGSGTSYSLTISRVEAEDAATYYCQQWSSNPPTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 19 - protein, 256 aa, Artificial Sequence
  Synthetic polypeptide
MGWSYIILFLVATATGVHSQVQLQQPGAELVKPGASVKLSCKASGYTFTSQWMHWVKQRPGQGLEWIGMIHPNSGSTNNNEKFKSKATLTVDKSSSTAYMQLSSLTSEDSAVYYCTRWAMDYWGQGTSVTASSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPK

SEQ ID NO: 20 - protein, 228 aa, Artificial Sequence
  Synthetic polypeptide
MKLPVRLLVLMFWIPASSTDVVMTQTPLSLPVSLGDLASISCRSSQSLVHSNGNTYLHWYLQKPGQSPKLLIFKVSNRFSGVPDRFSGSGSGTDFTLKISRVEAEDLGVYFCSQSTHVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 21 - protein, 291 aa, Artificial Sequence
  Synthetic polypeptide
MKLWLNWIFLVTLLNGIQCEVKLVESGGGLVQPGGSLSLSCAASGFTFTDYYMSWVRQPPGKALEWLGFIRHKAKGYTAEYSASVKGRFTISRDNSQSILYLQMNALRAEDSATYYCARLYYYGSPHWYFDVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 22 - protein, 225 aa, Artificial Sequence
  Synthetic polypeptide
MDFQVQIFSFLLISASVIISRGQIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGSGTELEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 23 - protein, 299 aa, Artificial Sequence
  Synthetic polypeptide
MGWSCIMLFLAATATGVHSQVQLQQPGAELVKPGASVKLSCKASDYTFTNYWMHWVKQRPGRGLEWIGRIDPSSGGAKYNEKFKSKATLTVDKPSSTAYMEFSSLTSEDSAVYYCVRSRYDYDGWFAYWGLGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDQ

SEQ ID NO: 24 - protein, 229 aa, Artificial Sequence
  Synthetic polypeptide
MRCLAEFLGLLVLWIPGAIGDIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 25 - protein, 285 aa, Artificial Sequence
  Synthetic polypeptide
MEWIWIFLFILSGTAGVQSQVQLQQSGAELARPGASVKLSCKASDDTFINYGINWVKQRTGQGLEWIGETFPSNGNTFYNEKFKGKATLTADRSSSTTYMELRSLTSEDAAVYFCARHSNLPYFDHWGQGSTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDQ

SEQ ID NO: 26 - protein, 230 aa, Artificial Sequence
  Synthetic polypeptide
MESQTQVLISLLFWVSGTCGDIVMTQSPSSLSVSAGEKVTMSCKSSQSLLNSGNQKNYLAWYQQKPGQPPKLLIYGASTRESGVPERFTGSGSGTDFTLTISSVQAEDLAVYYCQNDHSYPFTFGGGTNLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSP

SEQ ID NO: 27 - protein, 8 aa, Artificial Sequence
  Synthetic polypeptide
GYTFTSYW

SEQ ID NO: 28 - protein, 8 aa, Artificial Sequence
  Synthetic polypeptide
IIPSSGYT

SEQ ID NO: 29 - protein, 13 aa, Artificial Sequence
  Synthetic polypeptide
ARSDGSYPYYFDY

SEQ ID NO: 30 - protein, 5 aa, Artificial Sequence
  Synthetic polypeptide
SSVSY

SEQ ID NO: 31 - skipped

SEQ ID NO: 32 - protein, 9 aa, Artificial Sequence
  Synthetic polypeptide
FQGSGYPLT

SEQ ID NO: 33 - protein, 8 aa, Artificial Sequence
  Synthetic polypeptide
GYTFTSYD

SEQ ID NO: 34 - protein, 8 aa, Artificial Sequence
  Synthetic polypeptide
IYPRDGST

SEQ ID NO: 35 - protein, 13 aa, Artificial Sequence
  Synthetic polypeptide
ARGLWYYVSGMDY

SEQ ID NO: 36 - protein, 11 aa, Artificial Sequence
  Synthetic polypeptide
QSLVHSNGNTY

SEQ ID NO: 37 - skipped

SEQ ID NO: 38 - protein, 8 aa, Artificial Sequence
  Synthetic polypeptide
QSTHVPPT

SEQ ID NO: 39 - protein, 8 aa, Artificial Sequence
  Synthetic polypeptide
GYTFTSYW

SEQ ID NO: 40 - protein, 8 aa, Artificial Sequence
  Synthetic polypeptide
IIPSSGYT

SEQ ID NO: 41 - protein, 13 aa, Artificial Sequence
  Synthetic polypeptide
ARSTSGYPYYFDS

SEQ ID NO: 42 - protein, 5 aa, Artificial Sequence
  Synthetic polypeptide
SSVSF

SEQ ID NO: 43 - skipped

SEQ ID NO: 44 - protein, 9 aa, Artificial Sequence
  Synthetic polypeptide
FQGSWYPLT

SEQ ID NO: 45 - protein, 8 aa, Artificial Sequence
  Synthetic polypeptide
GYTFTTYW

SEQ ID NO: 46 - protein, 8 aa, Artificial Sequence
  Synthetic polypeptide
IIPSSGYS

SEQ ID NO: 47 - protein, 12 aa, Artificial Sequence
  Synthetic polypeptide
ARSTSGYPYYFD

SEQ ID NO: 48 - protein, 5 aa, Artificial Sequence
  Synthetic polypeptide
SSVSY

SEQ ID NO: 49 - skipped

SEQ ID NO: 50 - protein, 9 aa, Artificial Sequence
  Synthetic polypeptide
FQGSWYPLT

SEQ ID NO: 51 - protein, 8 aa, Artificial Sequence
  Synthetic polypeptide
GYTFTSYW

SEQ ID NO: 52 - protein, 8 aa, Artificial Sequence
  Synthetic polypeptide
IHPNSGST

SEQ ID NO: 53 - protein, 11 aa, Artificial Sequence
  Synthetic polypeptide
APRMFDDYDDY

SEQ ID NO: 54 - protein, 7 aa, Artificial Sequence
  Synthetic polypeptide
SSVSSSY

SEQ ID NO: 55 - skipped

SEQ ID NO: 56 - protein, 9 aa, Artificial Sequence
  Synthetic polypeptide
HHYHRSPYT

SEQ ID NO: 57 - protein, 8 aa, Artificial Sequence
  Synthetic polypeptide
GYTFTDYY

SEQ ID NO: 58 - protein, 8 aa, Artificial Sequence
  Synthetic polypeptide
INPNNGGT

SEQ ID NO: 59 - protein, 11 aa, Artificial Sequence
  Synthetic polypeptide
ARWGIYDRFTY

SEQ ID NO: 60 - protein, 6 aa, Artificial Sequence
  Synthetic polypeptide
QDINSF

SEQ ID NO: 61 - skipped

SEQ ID NO: 62 - protein, 9 aa, Artificial Sequence
  Synthetic polypeptide
LQYDEFPLT

SEQ ID NO: 63 - protein, 8 aa, Artificial Sequence
  Synthetic polypeptide
GYTFTSYD

SEQ ID NO: 64 - protein, 8 aa, Artificial Sequence
  Synthetic polypeptide
IYPSDGST

SEQ ID NO: 65 - protein, 13 aa, Artificial Sequence
  Synthetic polypeptide
ARGLWYYGGGVDY

SEQ ID NO: 66 - protein, 11 aa, Artificial Sequence
  Synthetic polypeptide
QSLEDSNGNTY

SEQ ID NO: 67 - skipped

SEQ ID NO: 68 - protein, 9 aa, Artificial Sequence
  Synthetic polypeptide
LQVTHVPYT

SEQ ID NO: 69 - protein, 8 aa, Artificial Sequence
  Synthetic polypeptide
GYTFTDYY

SEQ ID NO: 70 - protein, 8 aa, Artificial Sequence
  Synthetic polypeptide
INPNNGGT

SEQ ID NO: 71 - protein, 11 aa, Artificial Sequence
  Synthetic polypeptide
ARWGIFDRFTY

SEQ ID NO: 72 - protein, 6 aa, Artificial Sequence
  Synthetic polypeptide
QDINSF

SEQ ID NO: 73 - skipped

SEQ ID NO: 74 - protein, 9 aa, Artificial Sequence
  Synthetic polypeptide
LQYDEFPLT

SEQ ID NO: 75 - protein, 8 aa, Artificial Sequence
  Synthetic polypeptide
GYTFTDYN

SEQ ID NO: 76 - protein, 8 aa, Artificial Sequence
  Synthetic polypeptide
IYPNNGGT

SEQ ID NO: 77 - protein, 10 aa, Artificial Sequence
  Synthetic polypeptide
ARKTGTGFDY

SEQ ID NO: 78 - protein, 4 aa, Artificial Sequence
  Synthetic polypeptide
SVIY

SEQ ID NO: 79 - skipped

SEQ ID NO: 80 - protein, 9 aa, Artificial Sequence
  Synthetic polypeptide
QQWSSNPPT

SEQ ID NO: 81 - protein, 8 aa, Artificial Sequence
  Synthetic polypeptide
GYTFTSQW

SEQ ID NO: 82 - protein, 8 aa, Artificial Sequence
  Synthetic polypeptide
IHPNSGST

SEQ ID NO: 83 - protein, 7 aa, Artificial Sequence
  Synthetic polypeptide
TRWAMDY

SEQ ID NO: 84 - protein, 11 aa, Artificial Sequence
  Synthetic polypeptide
QSLVHSNGNTY

SEQ ID NO: 85 - skipped

SEQ ID NO: 86 - protein, 9 aa, Artificial Sequence
  Synthetic polypeptide
SQSTHVPWT

SEQ ID NO: 87 - protein, 8 aa, Artificial Sequence
  Synthetic polypeptide
GFTFTDYY

SEQ ID NO: 88 - protein, 10 aa, Artificial Sequence
  Synthetic polypeptide
IRHKAKGYTA

SEQ ID NO: 89 - protein, 15 aa, Artificial Sequence
  Synthetic polypeptide
ARLYYYGSPHWYFDV

SEQ ID NO: 90 - protein, 5 aa, Artificial Sequence
  Synthetic polypeptide
SSVSY

SEQ ID NO: 91 - skipped

SEQ ID NO: 92 - protein, 9 aa, Artificial Sequence
  Synthetic polypeptide
QQWSSNPPT

SEQ ID NO: 93 - protein, 8 aa, Artificial Sequence
  Synthetic polypeptide
DYTFTNYW

SEQ ID NO: 94 - protein, 8 aa, Artificial Sequence
  Synthetic polypeptide
IDPSSGGA

SEQ ID NO: 95 - protein, 13 aa, Artificial Sequence
  Synthetic polypeptide
VRSRYDYDGWFAY

SEQ ID NO: 96 - protein, 13 aa, Artificial Sequence
  Synthetic polypeptide
KSLLHSNGNTYLY

SEQ ID NO: 97 - skipped

SEQ ID NO: 98 - protein, 9 aa, Artificial Sequence
  Synthetic polypeptide
MQHLEYPFT

SEQ ID NO: 99 - protein, 8 aa, Artificial Sequence
  Synthetic polypeptide
DDTFINYG

SEQ ID NO: 100 - protein, 8 aa, Artificial Sequence
  Synthetic polypeptide
TFPSNGNT

SEQ ID NO: 101 - protein, 11 aa, Artificial Sequence
  Synthetic polypeptide
ARHSNLPYFDH

SEQ ID NO: 102 - protein, 12 aa, Artificial Sequence
  Synthetic polypeptide
QSLLNSGNQKNY

SEQ ID NO: 103 - skipped

SEQ ID NO: 104 - protein, 9 aa, Artificial Sequence
  Synthetic polypeptide
QNDHSYPFT